(19) United States
(12) Patent Application Publication
(10) Pub. No.: US 2005/0281743 A1
(43) Pub. Date: Dec. 22, 2005

(54) Cancer associated protein phosphatases and their uses

(76) Inventor: Allen D. Delaney, Vancouver (CA)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/509,773

(22) PCT Filed: Mar. 19, 2003
(86) PCT No.: PCT/CA03/00393

§ 371 Date: Apr. 14, 2005

(30) Foreign Application Priority Data
Mar. 28, 2002 (US) 6068859

(51) Int. Cl. A61K 49/00; G01N 33/574; C07K 14/82

(52) U.S. Cl. 424/9.2; 435/7.23; 530/350; 424/9.34; 530/400

(57) ABSTRACT

Detection of expression of the provided phosphatases in cancers is useful as a diagnostic, for determining the effectiveness of drugs, and for determining patient prognosis. The encoded polypeptides further provide a target for screening pharmaceutical agents effective in inhibiting the growth or metastasis of tumor cells. The present invention further provides methods and compositions relating to agents that specifically bind to MKPX, PTP4A1, PTPN7, FEM-2, DKFZP66K0524 or FLJ20313 for treatment and visualization of tumors in patients.

### BACKGROUND OF THE INVENTION

An accumulation of genetic changes underlies the development and progression of cancer, resulting in cells that differ from normal cells in their behavior, biochemistry, genetics, and microscopic appearance. Mutations in DNA that cause changes in the expression level of key proteins, or in the biological activity of proteins, are thought to be at the heart of cancer. For example, cancer can be triggered when genes that play a critical role in the regulation of cell division undergo mutations that lead to their over-expression. “Oncogenes” are involved in the dysregulation of growth that occurs in cancers. An aspect of oncogenesis that is often linked to tumor growth is angiogenesis. The growth of new blood vessels is essential for the later stages of solid tumor growth. Angiogenesis is caused by the migration and proliferation of the endothelial cells that form blood vessels.

Oncogene activity may involve kinases and phosphatases, enzymes that help regulate many cellular activities, particularly signaling from the cell membrane to the nucleus to initiate the cell's entrance into the cell cycle and to control other functions. These signaling pathways may involve kinases and phosphatases of proteins, or kinases or phosphatases of phosphatidylinositol (PI) lipids. PI is unique among membrane lipids because it can undergo reversible phosphorylation at multiple sites to generate a variety of distinct inositol phospholipids which participate in many aspects In the development, in particular in promoting cell survival and growth. Thus many kinases and phosphatases that are involved in regulating the generation of inositol phospholipids are likely to participate in oncogenesis.

Oncogenes may be tumor susceptibility genes, which are typically up-regulated in tumor cells, or may be tumor suppressor genes, which are down-regulated or absent in tumor cells. Malignancies can arise when a tumor suppressor is lost and/or an oncogene is inappropriately activated. When such mutations occur in somatic cells, they result in the growth of sporadic tumors.

Hundreds of genes have been implicated in cancer, but in most cases relationships between these genes and their effects are poorly understood. Using massively parallel gene expression analysis, scientists can now begin to connect these genes into related pathways.

Phosphorylation is Important in signal transduction mediated by receptors via extracellular biological signals such as growth factors or hormones. For example, many oncogenes are kinases or phosphatases, i.e. enzymes that catalyze protein phosphorylation or dephosphorylation reactions or are specifically regulated by phosphorylation. In addition, a kinase or phosphatase can have its activity regulated by one or more distinct kinase or phosphatases, resulting in specific signaling cascades.

Cloning procedures aided by homology searches of expressed sequence tag (EST) databases have accelerated the pace of discovery of new genes, but EST database searching remains an involved and onerous task. More than 3.6 million human EST sequences have been deposited in public databases, making it difficult to identify ESTs that represent new genes. Compounding the problems of scale are difficulties in detection associated with a high sequencing error rate and low sequence similarity between distant homologues.

Despite a long-felt need to understand and discover methods for regulating cells involved in various disease states, the complexity of signal transduction pathways has been a barrier to the development of products and processes for such regulation. Accordingly, there is a need in the art for improved methods for detecting and modulating the activity of such genes, and for treating diseases associated with the cancer and signal transduction pathways.

### RELEVANT LITERATURE

The use of genomic sequence in data mining for signaling proteins Is discussed in Schultz et al. Nature Genetics (2000) 25:201. Serine/threonine kinases and phosphatases have been reviewed, for example, by Cross T G et al. Exp Cell Res (2000) 256(1):34-41. PI signaling pathways are reviewed, for example, by Irvine in Curr. Opin. Cell Bio. (1992) 4:212-219.

### SUMMARY OF THE INVENTION

Several protein and phosphatidylinositol lipid phosphatases are herein shown to be over-expressed In hyper-proliferative cells. Detection of expression In hyper-proliferative cells is useful as a diagnostic; for determining the effectiveness and mechanism of action of therapeutic drug candidates, and for determining patient prognosis. These phosphatase sequences further provide a target for screening pharmaceutical agents effective in treating hyper-proliferative disorders. In a further embodiment, the present invention provides methods and compositions relating to agents, particularly antibodies that specifically bind to the phosphatase proteins, for treatment and visualization of hyper-proliferative disorders in patients.

### DETAILED DESCRIPTION

The MKPX, PTP4A1, PTPN7, FEM-2 (formerly KIAA0015), DKFZP566K0524 and FLJ20313 phosphatases are shown to be over-expressed in cancer cells. The encoded polypeptides provide targets for drug screening or altering expression levels, and for determining other molecular targets In phosphatase signal transduction pathways involved in transformation and growth of tumor cells. Detection of over-expression In cancers provides a useful diagnostic for predicting patient prognosis and probability of drug effectiveness. The present invention further provides methods and compositions relating to agents that specifically bind to these phosphatases, for treatment and visualization of tumors in patients.

### Phosphatases

The human cDNA sequences encoding MKPX, PTP4A1, PTPN7, FEM-2, DKFZP566K0524 and FLJ20313 are provided as SEQ ID NOS:1, 3, 5, 7, 9 and 11 respectively and the encoded polypeptide product is provided as SEQ ID NOS:2, 4, 6, 8, 10 and 12 respectively. Dot blot analysis of probes prepared from mRNA of tumors showed that expression of these genes are up-regulated In clinical samples of human tumors.

MKPX phosphatase. Activated mitogen-activated protein (MAP) kinases play an essential role controlling many cell division functions. Dual specificity protein phosphatases elicit selective inactivation of MAP kinases and are under tight transcriptional control. MKPX phosphatase Is dual-specific protein phosphatase. The open reading frame of MKPX predicts a protein of 184 amino acids related to the Vaccinia virus VH1 and human VH1-related (VHR) phosphatases. Expression VHR-related MKPX is highest In thymus, but also detectable in monocytes and lymphocytes. A MKPX-specific antiserum detects a protein with an apparent molecular mass of 19 kDa in many cells, Including T lymphocytes and monocytes. MKPX expression was not Induced by T cell activation, but decreased somewhat at later time points. In vitro, MKPX dephosphorylated the Erk2 mitogen-activated protein kinase with faster kinetics than did VHR, which is thought to be specific for Erk1 and 2. When expressed In Jurkat T cells, MKPX has the capacity to suppress T cell antigen receptor-Induced activation of Erk2 and of an NFAT/AP-1 luciferase reporter, but not an NF-[kappa]B reporter. MKPX is a member of the VH1/VHR group of small dual-specific phosphatases that act in mitogen-activated protein kinase signaling pathways (Alonso et al. J Biol Chem (2002) 277:5524-5528).

PTP4A1, otherwise known as protein tyrosine phosphatase IVA member 1 or PRL-1 is similar to the rat PRL-1 gene. Expression of the rat PRL-1 gene, which encodes a unique nuclear protein tyrosine phosphatase, is positively associated with cellular growth during liver development, regeneration, and oncogenesis but with differentiation in intestine and other tissues. The human PRL-1 gene is localized to chromosome 6 within band q12. Human, rat, and mouse PRL-1 are 100% conserved at the amino acid level and 55% identical to a newly identified Caenorhabditis elegans PRL-1. Two promoter activities, P1 and P2, are present in the human PRL-1 gene. An enhancer that bound a developmentally regulated factor, PRL-1 intron enhancer complex (PIEC), was localized to the first intron of the human PRL-1 gene. The presence of PIEC correlates with the ability of the intron enhancer to confer transcriptional activation in HepG2 and F9 cells. The intron enhancer contributes significantly to PRL-1 promoter activity in HepG2 cells which contain PIEC but not to NIH 3T3 cells which do not (Peng et al. (1998) J. Biol. Chem. 273 (27): 17286-17295) PTPN7. Protein tyrosine phosphatase, non-receptor type 7 (PTPN7) is involved in lymphocyte development and signal transduction. Tyrosine phosphorylation and dephosphorylating events have been shown to be central to the process of growth regulation and signal transduction. PTPN7 contains a tyrosine phosphatase domain and is expressed exclusively in thymus and spleen. A cDNA of 2760 bp encodes a 339-amino add, intracellular, single-domain tyrosine phosphatase. When expressed as a glutathionine-S-transferase fusion protein, efficient lysis of p-nitrophenyl phosphate Is noted, indicating in vitro enzymatic activity of the cloned gene product. Normal mouse lymphocytes increase mRNA expression 10-15-fold upon simulation with phytohemagglutinin, concanavalin A, lipopolysaccharide or anti-CD3 monoclonal antibody. This hematopoietic tyrosine phosphatase may play a role in the regulation of T and B lymphocyte development and signal transduction (Zanke et al., Eur J Immunol (1992) 22:235-9).

FEM-2. FEM-2, formerly known as KIAA0015, represents Is thought to be a Ca2+/calmodulin-dependent protein kinase phosphatases that promote apoptosis (Tan et al. J Biol Chem (2001) 276(47):44193-202). In Caenorhabditis elegans, fem-2, fem-2, and fem-3 play pivotal roles in sex determination. A mammalian homologue of the C. elegans sex-determining protein FEM-1, F1Aalpha, has been described. Although there is little evidence to link F1Aalpha to sex determination, F1Aalpha and FEM-1 both promote apoptosis in mammalian cells. Human FEM2 (hFEM-2) is similar to C. elegans FEM-2 and exhibits PP2C phosphatase activity and associates with FEM-3. hFEM-2 shows striking similarity (79% amino acid identity) to rat Ca(2+)/calmodulin (CaM)-dependent protein kinase phosphatase (rCaMKPase). hFEM-2 and FEM-2, but not PP2Calpha, were demonstrated to dephosphorylate CaM kinase II efficiently in vitro, suggesting that hFEM-2 is a specific phosphatase for CaM kinase. Furthermore, hFEM-2 and FEM-2 associated with F1Aalpha and FEM-1 respectively. Overexpression of hFEM-2, FEM-2, or rCaMKPase all mediated apoptosis in mammalian cells. The catalytically active, but not the Inactive, forms of hFEM-2 induced caspase-dependent apoptosis, which was blocked by Bd-XL or a dominant negative mutant of caspase-9. Human FEM-2 is likely to be a conserved CaM kinase phosphatases that plays a role in apoptosis signaling.

DKFZP566K0524. The function of human sequence DKFZP566K0524 is not known. However it is related to the protein tyrosine phosphatase, non-receptor type 20 gene of mice (Ohsugi et al. (1997) J Biol Chem 272:33092-9). This gene encodes a protein-tyrosine phosphatase expressed exclusively in mice tests. The gene encodes an open reading frame of 426 amino acids containing a single catalytic domain in the carboxyl-terminal half. Indirect immunofluorescence studies and in situ hybridization analysis showed that this protein was specifically expressed In testicular germ cells that have undergone meiosis. Developmentally, the mouse protein is detected between 2 and 3 weeks after birth, In parallel with the onset of meiosis. The mouse protein Is a member of the cytoplasmic protein-tyrosine phosphatases that may play an important role(s) in spermatogenesis and/or meiosis (Ohsugi et al. J Biol Chem (1997) 272:33092-9).

FLJ20313. However, FLJ20313 shows similarity to the phosphatidylinositol-3 phosphate 3-phosphatase adaptor subunit. D3-phosphoinositides act as second messengers by recruiting, and thereby activating, diverse signaling proteins. The rat phosphatidylinositol 3-phosphate [Ptdlns(3)P] 3-phosphatase, comprising a heterodimer of a 78-kDa adapter subunit in complex with a 65-kDa catalytic subunit. The human 3-phosphatase adapter subunit (3-PAP) shares significant sequence similarity with the protein and lipid 3-phosphatase myotubularin, and with several other members of the myotubularin gene family Including SET-binding factor 1. However, unlike myotubularin, 3-PAP does not contain a consensus HCX(5)R catalytic motif. The 3-PAP sequence contains several motifs that predict interaction with proteins containing Src homology-2 (SH2) domains, phosphotyrosine-binding (PTB) domains, members of the 14-3-3 family, as well as proteins with SET domains. Northern blot analysis identified two transcripts (5.5 kb and 2.5 kb) with highest abundance in human liver, kidney, lung, and placenta. 3-PAP immunoprecipitates isolated from platelet cytosol hydrolyzed the D3-phosphate from Ptdlns(3)P and Ptdlns 3,4-bisphosphate [Ptdlns(3,4)P(2)]. However, insect cell-expressed 3-PAP recombinant protein was catalytically inactive. The 3-PAP polypeptide may therefore be an adapter subunit (Nandurkar et al Proc Natl Acad Sci USA (2001) 98(17):9499-504).

### Hyper-Proliferative Disorders of Interest

The subject genes are used to diagnose a hyper-proliferative disorder, or their activities manipulated to treat a hyperproliferative disorders, e.g. to inhibit tumor growth, to inhibit angiogenesis, to decrease inflammation associated with a lymphoproliferative disorder, to inhibit graft rejection, or neurological damage due to tissue repair, etc. There are many disorders associated with a dysregulation of cellular proliferation. The conditions of interest include, but are not limited to, the following conditions.

The subject methods are applied to the treatment of a variety of conditions where there is proliferation and/or migration of smooth muscle cells, and/or inflammatory cells into the intimal layer of a vessel, resulting in restricted blood flow through that vessel, i.e. neointimal occlusive lesions. Occlusive vascular conditions of interest Include atherosclerosis, graft coronary vascular disease after transplantation, vein graft stenosis, peri-anastomatic prosthetic graft stenosis, restenosis after angioplasty or stent placement, and the like.

Diseases where there is hyperproliferation and tissue remodeling or repair of reproductive tissue, e.g. uterine, testicular and ovarian carcinomas, endometriosis, squamous and glandular epithelial carcinomas of the cervix, etc. are reduced In cell number by administration of the subject compounds Tumor cells are characterized by uncontrolled growth, Invasion to surrounding tissues, and metastatic spread to distant sites. Growth and expansion requires an ability not only to proliferate, but also to down-modulate cell death (apoptosis) and activate angiogenesis to produce a tumor neovasculature. Angiogenesis may be inhibited by affecting the cellular ability to Interact with the extracellular environment and to migrate, which is an integrin-specific function, or by regulating apoptosis of the endothelial cells. Integrins function in cell-to-cell and cell-to-extracellular matrix (ECM) adhesive interactions and transduce signals from the ECM to the cell interior and vice versa. Since these properties Implicate integrin involvement In cell migration, invasion, intra- and extra-vasation, and platelet interaction, a role for Integrins in tumor growth and metastasis is obvious.

Tumors of interest for treatment include carcinomas, e.g. colon, duodenal, prostate, ovarian, breast, melanoma, ductal, hepatic, pancreatic, renal, endometrial, stomach, dysplastic oral mucosa, polyposis, Invasive oral cancer, non-small cell lung carcinoma, transitional and squamous cell urinary carcinoma etc.; neurological malignancies, e.g. neuroblastoma, gliomas, etc.; hematological malignancies, e.g. childhood acute leukaemia, non-Hodgkin's lymphomas, chronic lymphocytic leukaemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; and the like.

Some cancers of particular Interest include breast cancers, which are primarily adenocarcinoma subtypes. Ductal carcinoma In situ is the most common type of noninvasive breast cancer. In DCIS, the malignant cells have not metastasized through the walls of the ducts into the fatty tissue of the breast. Infiltrating (or invasive) ductal carcinoma (IDC) has metastasized through the wall of the duct and invaded the fatty issue of the breast. Infiltrating (or invasive) lobular carcinema (ILC) is similar to IDC, in that it has the potential metastasize elsewhere in the body. About 10% to 15% of invasive breast cancers are invasive lobular carcinomas.

Also of interest is non-small cell lung carcinoma. Non-small cell lung cancer (NSCLC) is made up of three general subtypes of lung cancer. Epidermoid carcinoma (also called squamous cell carcinoma) usually starts In one of the larger bronchial tubes and grows relatively slowly. The size of these tumors can range from very small to quite large. Adenocarcinoma starts growing near the outside surface of the lung and may vary in both size and growth rate. Some slowly growing adenocarcinomas are described as alveolar cell cancer. Large cell carcinoma starts near the surface of the lung, grows rapidly, and the growth Is usually fairly large when diagnosed. Other less common forms of lung cancer are carcinold, cylindroma, mucoepidermold, and malignant mesothelloma.

Melanoma is a malignant tumor of melanocytes. Although most melanomas arise in the skin, they also may arise from mucosal surfaces or at other sites to which neural crest cells migrate. Melanoma occurs predominantly in adults, and more than half of the cases arise in apparently normal areas of the skin. Prognosis is affected by clinical and histological factors and by anatomic location of the lesion. Thickness and/or level of invasion of the melanoma, mitotic index, tumor infiltrating lymphocytes, and ulceration or bleeding at the primary site affect the prognosis. Clinical staging is based on whether the tumor has spread to regional lymph nodes or distant sites. For disease clinically confined to the primary site, the greater the thickness and depth of local invasion of the melanoma, the higher the chance of lymph node metastases and the worse the prognosis. Melanoma can spread by local extension (through lymphatics) and/or by hematogenous routes to distant sites. Any organ may be involved by metastases, but lungs and liver are common sites.

Other hyperproliferative diseases of interest relate to epidermal hyperproliferation, tissue remodeling and repair. For example, the chronic skin inflammation of psoriasis is associated with hyperplastic epidermal keratinocytes as well as infiltrating mononuclear cells, including CD4+ memory T cells, neutrophils and macrophages.

The proliferation of immune cells is associated with a number of autoimmune and lymphoproliferative disorders. Diseases of interest include multiple sclerosis, rheumatoid arthritis and insulin dependent diabetes mellitus. Evidence suggests that abnormalities in apoptosis play a part in the pathogenesis of systemic lupus erythematosus (SLE). Other lymphoproliferative conditions the Inherited disorder of lymphocyte apoptosis, which is an autoimmune lymphoproliferative syndrome, as well as a number of leukemias and lymphomas. Symptoms of allergies to environmental and food agents, as well as inflammatory bowel disease, may also be alleviated by the compounds of the invention.

Conditions treatable by inhibiting a molecule of the invention also include those associated with defects in cell cyde regulation or in response to extracellular signals, e.g. hyperglycemia and diabetes Type I and Type II, immunological disorders, e.g. autoimmune and immunodeficiency diseases; hyperproliferative disorders, which may include psoriasis, arthritis, inflammation, angiogenesis, endometriosis, scarring, cancer, etc.

### Diagnostic Applications

Determination of the presence of MKPX, PTP4A1, PTPN7, FEM-2, DKFZP566K0524 or FLJ20313 is used in the diagnosis, typing and staging of tumors. Detection of the presence of these phosphatases is performed by the use of a specific binding pair member to quantitate the specific protein, DNA or RNA present in a patient sample. Generally the sample will be a biopsy or other cell sample from the tumor. Where the tumor has metastasized, blood samples may be analyzed. MKPX, PTP4A1, PTPN7, FEM-2, DKFZP566K0524 or FLJ20313 can be used in screening methods to identify candidate therapeutic agents and other therapeutic targets. Methods providing agents that bind to these proteins are provided as cancer treatments and for cancer imaging.

In a typical assay, a tissue sample, e.g. biopsy, blood sample, etc. is assayed for the presence of MKPX, PTP4A1, PTPN7, FEM-2, DKFZP566K0524 or FLJ20313 specific sequences by combining the sample with a specific binding member, and detecting directly or indirectly the presence of the complex formed between the two members. The term “specific binding member” as used herein refers to a member of a specific binding pair, i.e. two molecules where one of the molecules through chemical or physical means specifically binds to the other molecule. One of the molecules will be a nucleic add e.g. corresponding to SEQ ID NOS:1, 3, 5, 7, 9 or 11, or a polypeptide encoded by the nucleic acid, which can include any protein substantially similar to the proteins or a fragment thereof; or any nucleic acid substantially similar to the nucleotide sequence provided in SEQ ID NOS:1, 3, 5, 7, 9 or 11 or a fragment thereof. The complementary members of a specific binding pair are sometimes referred to as a ligand and receptor.

Binding pairs of interest include antigen and antibody specific binding pairs, peptide-MHC antigen and T-cell receptor pairs; complementary nucleotide sequences (including nucleic acid sequences used as probes and capture agents in DNA hybridization assays); phosphatase protein and substrate pairs; autologous monoclonal antibodies, and the like. The specific binding pairs may include analogs, derivatives and fragments of the original specific binding member. For example, an antibody directed to a protein antigen may also recognize peptide fragments, chemically synthesized peptidomimetics, labeled protein, derivatized protein, etc. so long as an epitope is present.

Nucleic acid sequences. Nucleic acids encoding MKPX, PTP4A1, PTPN7, FEM-2, DKFZP566K0524 or FLJ20313 are useful in the methods of the invention, e.g. as a specific binding member, to produce the encoded polypeptide, etc. The nucleic acids of the invention also include nucleic acids having a high degree of sequence similarity or sequence identity to SEQ ID NOS:1, 3, 5, 7, 9 or 11. Sequence identity can be determined by hybridization under stringent conditions, for example, at 50° C. or higher and 0.1×SSC (9 mM saline/0.9 mM sodium citrate). Hybridization methods and conditions are well known In the art, see, e.g., U.S. Pat. No. 5,707,829. Nucleic acids that are substantially identical to the provided nucleic acid sequence, e.g. allelic variants, genetically altered versions of the gene, etc., bind to SEQ ID NOS:1, 3, 5, 7, 9 or 11 under stringent hybridization conditions.

The nucleic acids can be cDNAs or genomic DNAs, as well as fragments thereof. The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide of the Invention.

A genomic sequence of Interest comprises the nucleic add present between the initiation codon and the stop codon, as defined In the listed sequences, Including all of the introns that are normally present in a native chromosome. It can further include the 3′ and 5′ untranslated regions found In the mature mRNA. It can further include specific transcriptional and translational regulatory sequences, such as promoters, enhancers, etc., including about 1 kb, but possibly more, of flanking genomic DNA at either the 5′ or 3′ end of the transcribed region. The genomic DNA flanking the coding region, either 3′ or 5′, or internal regulatory sequences as sometimes found In introns, contains sequences required for proper tissue, stage-specific, or disease-state specific expression, and are useful for investigating the up-regulation of expression In tumor cells.

Probes specific to the nucleic acid of the invention can be generated using an nucleic acid sequence, e.g. as disclosed in SEQ ID NOS:1, 3, 5, 7, 9 or 11. The probes are preferably at least about 18 nt, 25 nt, 50 nt or more of the corresponding contiguous, and are usually less than about 2, 1, or 0.5 kb in length. Preferably, probes are designed based on a contiguous sequence that remains unmasked following application of a masking program for masking low complexity, e.g. BLASTX. Double or single stranded fragments can be obtained from the DNA sequence by chemically synthesizing oligonucleotides in accordance with conventional methods, by restriction enzyme-digestion, by PCR amplification, etc. The probes can be labeled, for example, with a radioactive, biotinylated, or fluorescent tag.

The nucleic adds of the subject invention are isolated and obtained in substantial purity, generally as other than an Intact chromosome. Usually, the nucleic acids, either as DNA or RNA, will be obtained substantially free of other naturally-occurring nucleic add sequences, generally being at least about 50%, usually at least about 90% pure and are typically “recombinant,” e.g., flanked by one or more nucleotides with which it is not normally associated on a naturally occurring chromosome.

The nucleic acids of the invention can be provided as a linear molecule or within a circular molecule, and can be provided within autonomously replicating molecules (vectors) or within molecules without replication sequences. Expression of the nucleic acids can be regulated by their own or by other regulatory sequences known In the art. The nucleic acids of the invention can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other. For hybridization probes, it may be desirable to use nucleic acid analogs, in order to improve the stability and binding affinity. The term “nucleic acid” shall be understood to encompass such analogs.

Polypeptide Compositions. The present invention further provides polypeptides encoded by SEQ ID NOS:1, 3, 5, 7, 9 and 11 and variants thereof, which can be used for a variety of purposes. The polypeptides contemplated by the invention include those encoded by the disclosed nucleic acids, as well as nucleic acids that, by virtue of the degeneracy of the genetic code, are not identical In sequence to the disclosed nucleic adds, and vairants thereof.

In general, the term “polypeptide” as used herein refers to both the full length polypeptide encoded by the recited nucleic acid, the polypeptide encoded by the gene represented by the recited nucleic acid, as well as portions or fragments thereof. “Polypeptides” also includes variants of the naturally occurring proteins, where such variants are homologous or substantially similar to the naturally occurring protein, and can be of an origin of the same or different species as the naturally occurring protein (e.g., human, murine, or some other species that naturally expresses the recited polypeptide, usually a mammalian species). In general, variant polypeptides have a sequence that has at least about 80%, usually at least about 90%, and more usually at least about 98% sequence identity with a differentially expressed polypeptide described herein, as measured by BLAST 2.0 using the parameters described above. The variant polypeptides can be naturally or non-naturally glycosylated, i.e., the polypeptide has a glycosylation pattern that differs from the glycosylation pattern found In the corresponding naturally occurring protein.

In general, the polypeptides of the subject Invention are provided in a non-naturally occurring environment, e.g. are separated from their naturally occurring environment. In certain embodiments, the subject protein Is present in a composition that is enriched for the protein as compared to a control. As such, purified polypeptides are provided, where by purified is meant that the protein is present in a composition that is substantially free of non-differentially expressed polypeptides, where by substantially free is meant that less than 90%, usually less than 60% and more usually less than 50% of the composition is made up of non-MKPX, PTP4A1, PTPN7, FEM-2, DKFZP566K0524 or FLJ20313 polypeptides.

Variant polypeptides can include amino acid substitutions, additions or deletions. The amino add substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino add substitutions are those that preserve the general charge, hydrophobicity/hydrophilicity, and/or steric bulk of the amino acid substituted. Variants can be designed so as to retain or have enhanced biological activity of a particular region of the protein (e.g., a functional domain and/or, where the polypeptide is a member of a protein family, a region associated with a consensus sequence).

Variants also include fragments of the polypeptides disclosed herein, particularly biologically active fragments and/or fragments corresponding to functional domains. Fragments of interest will typically be at least about 10 aa to at least about 15 aa in length, usually at least about 50 aa In length, and can be as long as 300 aa in length or longer, but will usually not exceed about 500 aa in length, where the fragment will have a contiguous stretch of amino acids that is identical to a polypeptide encoded by SEQ ID NOS:1, 3, 5, 7, 9 or 11, or a homolog thereof.

Antibodies. As used herein, the term “antibodies” Includes antibodies of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a green fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

“Antibody specificity”, in the context of antibody-antigen interactions, is a term well understood in the art, and indicates that a given antibody binds to a given antigen, wherein the binding can be inhibited by that antigen or an epitope thereof which is recognized by the antibody, and does not substantially bind to unrelated antigens. Methods of determining specific antibody binding are well known to those skilled in the art, and can be used to determine the specificity of antibodies of the invention for a polypeptide, particularly MKPX, PTP4A1, PTPN7, FEM-2, DKFZP566K0524 or FLJ20313.

As used herein, a compound which specifically binds to human protein MKPX, PTP4A1, PTPN7, FEM-2, DKFZP566K0524 or FLJ20313 is any compound (such as an antibody) which has a binding affinity for any naturally occurring isoform, splice variant, or polymorphism. As one of ordinary skill in the art will appreciate, such “specific” binding compounds (e.g., antibodies) may also bind to other closely related proteins which exhibit significant homology, for example, having greater than 90% identity, more preferably greater than 95% identity, and most preferably greater than 99% Identity with the amino acid sequence of SEQ ID NOS:2, 4, 6, 8, 10 or 12. Such proteins may Include truncated forms or domains of SEQ ID NOS:2, 4, 6, 8, 10 or 12, and recombinantly engineered alterations of SEQ ID NOS:2, 4, 6, 8, 10 or 12. For example, a portion of SEQ ID NOS:2, 4, 6, 8, 10 or 12 may be engineered to encode a non-naturally occurring cysteine for cross-linking to an immunoconjugate protein, as described below.

Selection of antibodies which alter (enhance or inhibit) the binding of a compound to MKPX, PTP4A1, PTPN7, FEM-2, DKFZP566K0524 or FLJ20313 may be accomplished by a straightforward binding inhibition/enhancement assay. According to standard techniques, the binding of a labeled (e.g., fluorescently or enzyme-labeled) antibody to a protein of the invention, which has been immobilized in a microtiter well, is assayed using standard phosphatase assays in both the presence and absence of the ligand. The change in binding is indicative of either an enhancer (increased binding) or competitive inhibitor (decreased binding) relationship between the antibody and the ligand. Such assays may be carried out in high-throughput formats (e.g., 384 well plate formats, in robotic systems) for the automated selection of monoclonal antibody candidates for use as ligand or substrate-binding inhibitors or enhancers.

In addition, antibodies that are useful for altering the function of a protein of the invention may be assayed in functional formats. In cell-based assays of activity, expression of a protein of the invention is first verified in the particular cell strain to be used. If necessary, the cell line may be stably transfected with a coding sequence under the control of an appropriate constituent promoter, in order to express a protein of the invention at a level comparable to that found in primary tumors. The ability of the tumor cells to survive in the presence of the candidate function-altering-antibody is then determined. Similarly, In vivo models for human cancer, particularly colon, pancreas, lung and ovarian cancer are available as nude mice/SCID mice or rats, have been described. Once expression of a protein of the invention in the tumor model Is verified, the effect of the candidate antibodies on the tumor masses in these models can evaluated, wherein the ability of the antibody candidates to alter phosphatase activity Is indicated by a decrease in tumor growth or a reduction in the tumor mass. Thus, antibodies that exhibit the appropriate anti-tumor effect may be selected without direct knowledge of a binding ligand.

Generally, as the term is utilized in the specification, “antibody” or “antibody moiety” is intended to include any polypeptide chain-containing molecular structure that has a specific shape which fits to and recognizes an epitope, where one or more non-covalent binding interactions stabilize the complex between the molecular structure and the epitope. Antibodies which bind specifically to a protein of the invention are referred to as anti-phosphatase antibodies. The specific or selective fit of a given structure and its specific epitope is sometimes referred to as a “lock and key” fit. The archetypal antibody molecule is the immunoglobulin, and all types of immunoglobulins (IgG, IgM, IgA, IgE, IgD, etc.), from all sources (e.g., human, rodent, rabbit, cow, sheep, pig, dog, other mammal, chicken, turkey, emu, other avians, etc.) are considered to be “antibodies.” Antibodies utilized in the present invention may be polyclonal antibodies, although monoclonal antibodies are preferred because they may be reproduced by cell culture or recombinantly, and may be modified to reduce their antigenicity.

Polyclonal antibodies may be raised by a standard protocol by injecting a production animal with an antigenic composition, formulated as described above. See, e.g., Harlow and Lane, Antibodies: A Laboratory Manual, Cold Spring Harbor Laboratory, 1988. In one such technique, an antigenic portion of a MKPX, PTP4A1, PTPN7, FEM-2, DKFZP566K0524 or FLJ20313 polypeptide is initially injected into any of a wide variety of mammals (e.g., mice, rats, rabbits, sheep or goats). Alternatively, in order to generate antibodies to relatively short peptide portions of MKPX, PTP4A1, PTPN7, FEM-2, DKFZP566K0524 or FLJ20313, a superior immune response may be elicited if the polypeptide is joined to an immunogenic carrier, such as ovalbumin, BSA, KLH, pre-S HBsAg, other viral or eukaryotic proteins, and the like. The peptide-conjugate is injected into the animal host, preferably according to a predetermined schedule Incorporating one or more booster immunizations, and the animals are bled periodically. Polyclonal antibodies specific for the polypeptide may then be purified from such anti-sera by, for example, affinity chromatography using the polypeptide coupled to a suitable solid support.

Alternatively, for monoclonal antibodies, hybridomas may be formed by isolating the stimulated Immune cells, such as those from the spleen of the Inoculated animal. These cells are then fused to immortalized cells, such as myeloma cells or transformed cells, which are capable of replicating indefinitely in cell culture, thereby producing an immortal, immunoglobulin-secreting cell line. The immortal cell line utilized is preferably selected to be deficient in enzymes necessary for the utilization of certain nutrients. Many such cell lines (such as myelomas) are known to those skilled in the art, and include, for example: thymidine phosphatase (TK) or hypoxanthine-guanine phosphoriboxyl transferase (HGPRT). These deficiencies allow selection for fused cells according to their ability to grow on, for example, hypoxanthine aminopterinthymidine medium (HAT).

Preferably, the immortal fusion partners utilized are derived from a line that does not secrete immunoglobulin. The resulting fused cells, or hybridomas, are cultured under conditions that allow for the survival of fused, but not unfused, cells and the resulting colonies screened for the production of the desired monoclonal antibodies. Colonies producing such antibodies are cloned, expanded, and grown so as to produce large quantities of antibody, see Kohler and Milstein, Nature (1975) 256:495 (the disclosure of which is herein Incorporated by reference).

Large quantities of monoclonal antibodies from the secreting hybridomas may then be produced by injecting the clones into the peritoneal cavity of mice and harvesting the ascites fluid therefrom. The mice, preferably primed with pristine, or some other tumor-promoter, and immunosuppressed chemically or by irradiation, may be any of various suitable strains known to those in the art. The ascites fluid is harvested from the mice and the monoclonal antibody purified therefrom, for example, by CM Sepharose column chromatography or other chromatographic means. Alternatively, the hybridomas may be cultured in vitro or as suspension cultures. Batch, continuous culture, or other suitable culture processes may be utilized. Monoclonal antibodies are then recovered from the culture medium or supernatant. It is preferred that such antibodies by humanized or chimerized according to one of the procedures outlined below.

In addition, the antibodies or antigen binding fragments may be produced by genetic engineering. In this technique, as with the standard hybridoma procedure, antibody-producing cells are sensitized to the desired antigen or immunogen. The messenger RNA Isolated from the immune spleen cells or hybridomas is used as a template to make cDNA using PCR amplification. A library of vectors, each containing one heavy chain gene and one light chain gene retaining the Initial antigen specificity, is produced by insertion of appropriate sections of the amplified immunoglobulin cDNA Into the expression vectors. A combinatorial library is constructed by combining the heavy chain gene library with the light chain gene library. This results in a library of clones which co-express a heavy and light chain (resembling the Fab fragment or antigen binding fragment of an antibody molecule). The vectors that carry these genes are co-transfected into a host (e.g. bacteria, insect cells, mammalian cells, or other suitable protein production host cell.). When antibody gene synthesis Is induced in the transfected host, the heavy and light chain proteins self-assemble to produce active antibodies that can be detected by screening with the antigen or immunogen.

Preferably, recombinant antibodies are produced in a recombinant protein production system which correctly glycosylates and processes the immunoglobulin chains, such as insect or mammalian cells, as is known in the art.

Antibodies that have a reduced propensity to induce a violent or detrimental immune response In humans (such as anaphylactic shock), and which also exhibit a reduced propensity for priming an immune response which would prevent repeated dosage with the antibody therapeutic or imaging agent (e.g., the human-anti-murine-antibody “HAMA” response), are preferred for use in the invention. Although some increased Immune response against the tumor is desirable, the concurrent binding and inactivation of the therapeutic or imaging agent generally outweighs this benefit Thus, humanized, chimeric, or xenogenic human antibodies, which produce less of an immune response when administered to humans, are preferred for use in the present invention.

Chimeric antibodies may be made by recombinant means by combining the murine variable light and heavy chain regions (VK and VH), obtained from a murine (or other animal-derived) hybridoma clone, with the human constant light and heavy chain regions, in order to produce an antibody with predominantly human domains. The production of such chimeric antibodies is well known in the art, and may be achieved by standard means (as described, e.g., in U.S. Pat. No. 5,624,659, incorporated fully herein by reference.) Humanized antibodies are engineered to contain even more human-like immunoglobulin domains, and incorporate only the complementarity-determining regions of the animal-derived antibody. This is accomplished by carefully examining the sequence of the hyper-variable loops of the variable regions of the monoclonal antibody, and fitting them to the structure of the human antibody chains. Although facially complex, the process is straightforward In practice. See, e.g., U.S. Pat. No. 6,187,287, incorporated fully herein by reference.

Alternatively, polyclonal or monoclonal antibodies may be produced from animals which have been genetically altered to produce human immunoglobulins, such as the Abgenix XenoMouse™ or the Medarex HuMAb® technology. The transgenic animal may be produced by initially producing a “knockout” animal which does not produce the animal's natural antibodies, and stably transforming the animal with a human antibody locus (e.g., by the use of a human artificial chromosome.) Only human antibodies are then made by the animal. Techniques for generating such animals, and deriving antibodies therefrom, are described in U.S. Pat. Nos. 6,162,963 and 6,150,584, Incorporated fully herein by reference.

Alternatively, single chain antibodies (Fv, as described below) can be produced from phage libraries containing human variable regions (described in e.g. U.S. Pat. No. 6,174,708, Incorporated fully herein by reference).

In addition to entire immunoglobulins (or their recombinant counterparts), immunoglobulin fragments comprising the epitope binding site (e.g., Fab′, F(ab′)₂, or other fragments) are useful as antibody moieties in the present Invention. Such antibody fragments may be generated from whole Immunoglobulins by ficin, pepsin, papain, or other protease cleavage. “Fragment,” or minimal Immunoglobulins may be designed utilizing 35′ recombinant immunoglobulin techniques. For instance “Fv” immunoglobulins for use In the present invention may be produced by linking a variable light chain region to a variable heavy chain region via a peptide linker (e.g., poly-glycine or another sequence which does not form an alpha helix or beta sheet motif).

Fv fragments are heterodimers of the variable heavy chain domain (V_{H}) and the variable light chain domain (V_{L}). The heterodimers of heavy and light chain domains that occur in whole IgG, for example, are connected by a disulfide bond. Recombinant Fvs in which V_{H} and V_{L} are connected by a peptide linker are typically stable, see, for example, Huston et al., Proc Natl Acad Sci USA (1988) 85:5879-5883 and Bird et al., Science (1988) 242:423-426, both fully incorporated herein, by reference. These are single chain Fvs which have been found to retain specificity and affinity and have been shown to be useful for imaging tumors and to make recombinant immunotoxins for tumor therapy. However, researchers have found that some of the single chain Fvs have a reduced affinity for antigen and the peptide linker can interfere with binding. Improved Fv's have also been made which comprise stabilizing disulfide bonds between the V_{H} and V_{L} regions, as described In U.S. Pat. No. 6,147,203, Incorporated fully herein by reference. Any of these minimal antibodies may be utilized in the present invention, and those which are humanized to avoid HAMA reactions are preferred for use In embodiments of the invention.

In addition, derivatized immunoglobulins with added chemical linkers, detectable moieties (fluorescent dyes, enzymes, substrates, chemiluminescent moieties), or specific binding moieties (such as streptavidin, avidin, or biotin) may be utilized in the methods and compositions of the present invention. For convenience, the term “antibody” or “antibody moiety” will be used throughout to generally refer to molecules which specifically bind to a MKPX, PTP4A1, PTPN7, FEM-2, DKFZP566K0524 or FLJ20313 epitope, although the term will encompass all immunoglobulins, derivatives, fragments, recombinant or engineered immunoglobulins, and modified immunoglobulins, as described above.

Candidate anti-phosphatase antibodies can be tested for activity by any suitable standard means. As a first screen, the antibodies may be tested for binding against the antigen utilized to produce them, or against the entire extracellular domain or protein. As a second screen, candidates may be tested for binding to an appropriate cell line, or to primary tumor tissue samples. For these screens, the candidate antibody may be labeled for detection (e.g., with fluorescein or another fluorescent moiety, or with an enzyme such as horseradish peroxidase). After selective binding is established, the candidate antibody, or an antibody conjugate produced as described below, may be tested for appropriate activity (i.e., the ability to decrease tumor cell growth and/or to aid in visualizing tumor cells) in an in vivo model, such as an appropriate cell line, or in a mouse or rat or mouse tumor model, as described above.

### Quantitation of Nucleic Acids

MKPX, PTP4A1, PTPN7, FEM-2, DKFZP566K0524 or FLJ20313 nucleic acid reagents are used to screen patent samples, e.g. biopsy-derived tumors, inflammatory samples such as arthritic synovium, etc., for amplified DNA in the cell, or increased expression of the corresponding mRNA or protein. DNA-based reagents are also designed for evaluation of chromosomal loci implicated in certain diseases e.g. for use in loss-of-heterozygosity (LOH) studies, or design of primers based on coding sequences.

The polynucleotides of the invention can be used to detect differences in expression levels between two cells, e.g., as a method to identify abnormal or diseased tissue in a human. The tissue suspected of being abnormal or diseased can be derived from a different tissue type of the human, but preferably it is derived from the same tissue type; for example, an intestinal polyp or other abnormal growth should be compared with normal intestinal tissue. The normal tissue can be the same tissue as that of the test sample, or any normal tissue of the patient, especially those that express the polynucleotide-related gene of interest (e.g., brain, thymus, testis, heart, prostate, placenta, spleen, small intestine, skeletal muscle, pancreas, and the mucosal lining of the colon, etc.). A difference between the polynucleotide-related gene, mRNA, or protein in the two tissues which are compared, for example, in molecular weight, amino acid or nucleotide sequence, or relative abundance, indicates a change in the gene, or a gene which regulates it, In the tissue of the human that was suspected of being diseased.

The subject nucleic acid and/or polypeptide compositions may be used to analyze a patient sample for the presence of polymorphisms associated with a disease state. Biochemical studies may be performed to determine whether a sequence polymorphism in a coding region or control region is associated with disease, particularly cancers and other growth abnormalities. Diseases of interest may also include other hyperproliferative disorders. Disease associated polymorphisms may include deletion or truncation of the gene, mutations that alter expression level, that affect the binding activity of the protein, the phosphatase activity domain, etc.

Changes in the promoter or enhancer sequence that may affect expression levels can be compared to expression levels of the normal allele by various methods known in the art. Methods for determining promoter or enhancer strength include quantitation of the expressed natural protein; insertion of the variant control element Into a vector with a reporter gene such as beta-galactosidase, luciferase, chloramphenicol acetyltransferase, etc. that provides for convenient quantitation; and the like.

A number of methods are available for analyzing nucleic acids for the presence of a specific sequence, e.g. upregulated expression. Cells that express MKPX, PTP4A1, PTPN7, FEM-2, DKFZP566K0524 or FLJ20313 may be used as a source of mRNA, which may be assayed directly or reverse transcribed into cDNA for analysis. The nucleic acid may be amplified by conventional techniques, such as the polymerase chain reaction (PCR), to provide sufficient amounts for analysis. The use of the polymerase chain reaction is described In Saiki et al. Science (1985) 239:487, and a review of techniques may be found in Sambrook et al. Molecular Cloning: A Laboratory Manual, CSH Press 1989, pp. 14.2-14.33.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein(6-FAM), 2,7-dimethoxy-4,5-dichloro-6-carboxyfluorescein (JOE), 6-carboxy-X-rhodamine (ROX), 6-carboxy-2,4,7,4,7-hexachlorofluorescein (HEX), 5-carboxyfluorescein (5-FAM) or N,N,N,N-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified or cloned fragment, is analyzed by one of a number of methods known In the art. Probes may be hybridized to Northern or dot blots, or liquid hybridization reactions performed. The nucleic acid may be sequenced by dideoxy or other methods, and the sequence of bases compared to a wild-type sequence. Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Fractionation Is performed by gel or capillary electrophoresis, particularly acrylamide or agarose gels.

Arrays provide a high throughput technique that can assay a large number of polynucleotides In a sample. In one aspect of the invention, an array is constructed comprising MKPX, PTP4A1, PTPN7, FEM-2, DKFZP566K0524 or FLJ20313 In conjunction with other cancer associated sequences, particularly cancer associated phosphatases. This technology can be used as a tool to test for differential expression.

A variety of methods of producing arrays, as well as variations of these methods, are known in the art and contemplated for use in the Invention. For example, arrays can be created by spotting polynucleotide probes onto a substrate (e.g., glass, nitrocellulose, etc.) in a two-dimensional matrix or array having bound probes. The probes can be bound to the substrate by either covalent bonds or by non-specific interactions, such as hydrophobic interactions. Samples of nucleic acids can be detectably labeled (e.g., using radioactive or fluorescent labels) and then hybridized to the probes. Double stranded nucleic acids, comprising the labeled sample polynucleotides bound to probe nucleic acids, can be detected once the unbound portion of the sample is washed away. Alternatively, the nucleic acids of the test sample can be immobilized on the array, and the probes detectably labeled.

Techniques for constructing arrays and methods of using these arrays are described in, for example, Schena et al., Proc Natl Aced Sci USA (1996) 93(20):106149; Schena et al., Science (1995) 270(5235):467-70; Shalon et al., Genome Res (1996) 6(7):63945, U.S. Pat. Nos. 5,807,522; 5,593,839; 5,578,832; 5,631,734; 5,599,695; and 5,556,752; EP 799 897; WO 97/29212; WO 97/27317; EP 785 280; WO 97/02357; EP 728 520; EP 721 016; and WO 95/22058.

Arrays can be used to, for example, examine differential expression of genes and can be used to determine gene function. For example, arrays can be used to detect differential expression of SEQ ID NOS:1, 3, 5, 7, 9 or 11, where expression is compared between a test cell and control cell (e.g., cancer cells and normal cells). High expression of a particular message in a cancer cell, which Is not observed in a corresponding normal cell, indicates a cancer specific gene product. Exemplary uses of arrays are further described in, for example, Pappalarado et al., Sem Radiation Oncol (1998) 8:217; and Ramsay, Nature Biotechnol (1998) 16:40. Furthermore, many variations on methods of detection using arrays are well within the skill in the art and within the scope of the present Invention. For example, rather than immobilizing the probe to a solid support, the test sample can be immobilized on a solid support that Is then contacted with the probe.

### Polypeptide Analysis

Screening for expression of the subject sequences may be based on the functional or antigenic characteristics of the protein. Protein truncation assays are useful In detecting deletions that may affect the biological activity of the protein. Various Immunoassays designed to detect polymorphisms in MKPX, PTP4A1, PTPN7, FEM-2, DKFZP566K0524 or FLJ20313 may be used In screening. Where many diverse genetic mutations lead to a particular disease phenotype, functional protein assays have proven to be effective screening tools. The activity of the encoded protein in phosphatase assays, etc., may be determined by comparison with the wild-type protein.

A sample is taken from a patient with cancer. Samples, as used herein, include biological fluids such as blood; organ or tissue culture derived fluids; etc. Biopsy samples or other sources of carcinoma cells are of particular interest, e.g. tumor biopsy, etc. Also included in the term are derivatives and fractions of such cells and fluids. The number of cells in a sample will generally be at least about 103, usually at least 104, and may be about 105 or more. The cells may be dissociated, In the case of solid tissues, or tissue sections may be analyzed. Alternatively a lysate of the cells may be prepared.

Detection may utilize staining of cells or histological sections, performed in accordance with conventional methods. The antibodies or other specific binding members of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent Final detection uses a substrate that undergoes a color change In the presence of the peroxidase. The absence or presence of antibody binding may be determined by various methods, including flow cytometry of dissociated cells, microscopy, radiography, scintillation counting, etc.

An alternative method for diagnosis depends on the in vitro detection of binding between antibodies and the MKPX, PTP4A1, PTPN7, FEM-2, DKFZP566K0524 or FLJ20313 in a lysate. Measuring the concentration of the target protein in a sample or fraction thereof may be accomplished by a variety of specific assays. A conventional sandwich type assay may be used. For example, a sandwich assay may first attach specific antibodies to an insoluble surface or support. The particular manner of binding is not crucial so long as it is compatible with the reagents and overall methods of the invention. They may be bound to the plates covalently or non-covalently, preferably non-covalently.

The Insoluble supports may be any compositions to which polypeptides can be bound, which is readily separated from soluble material, and which is otherwise compatible with the overall method. The surface of such supports may be solid or porous and of any convenient shape. Examples of suitable insoluble supports to which the receptor is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

Patient sample lysates are then added to separately assayable supports (for example, separate wells of a microtiter plate) containing antibodies. Preferably, a series of standards, containing known concentrations of the test protein is assayed in parallel with the samples or aliquots thereof to serve as controls. Preferably, each sample and standard will be added to multiple wells so that mean values can be obtained for each. The incubation time should be sufficient for binding, generally, from about 0.1 to 3 hr is sufficient. After incubation, the Insoluble support is generally washed of non-bound components. Generally, a dilute non-ionic detergent medium at an appropriate pH, generally 7-8, is used as a wash medium. From one to six washes may be employed, with sufficient volume to thoroughly wash non-specifically bound proteins present in the sample.

After washing, a solution containing a second antibody is applied. The antibody will bind to a polypeptide of the Invention with sufficient specificity such that it can be distinguished from other components present. The second antibodies may be labeled to facilitate direct, or indirect quantification of binding. Examples of labels that permit direct measurement of second receptor binding include radiolabels, such as ³H or ¹²⁵I, fluorescers, dyes, beads, chemilumninescers, colloidal particles, and the like. Examples of labels that permit indirect measurement of binding include enzymes where the substrate may provide for a colored or fluorescent product In a preferred embodiment, the antibodies are labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. The Incubation time should be sufficient for the labeled ligand to bind available molecules. Generally, from about 0.1 to 3 hr Is sufficient, usually 1 hr sufficing.

After the second binding step, the insoluble support is again washed free of non-specifically bound material, leaving the specific complex formed between the target protein and the specific binding member. The signal produced by the bound conjugate Is detected by conventional means. Where an enzyme conjugate is used, an appropriate enzyme substrate is provided so a detectable product Is formed.

Other immunoassays are known in the art and may find use as diagnostics. Ouchterlony plates provide a simple determination of antibody binding. Western blots may be performed on protein gels or protein spots on filters, using a detection system specific for one of the proteins of the invention as desired, conveniently using a labeling method as described for the sandwich assay.

In some cases, a competitive assay will be used. In addition to the patient sample, a competitor to the targeted protein is added to the reaction mix. The competitor and the selected phosphatase compete for binding to the specific binding partner. Usually, the competitor molecule will be labeled and detected as previously described, where the amount of competitor binding will be proportional to the amount of target protein present The concentration of competitor molecule will be from about 10 times the maximum anticipated protein concentration to about equal concentration in order to make the most sensitive and linear range of detection.

In some embodiments, the methods are adapted for use in vivo, e.g., to locate or identify sites where cancer cells are present. In these embodiments, a detectably-labeled moiety, e.g., an antibody, which Is specific for MKPX, PTP4A1, PTPN7, FEM-2, DKFZP566K0524 or FLJ20313 is administered to an individual (e.g., by injection), and labeled cells are located using standard Imaging techniques, including, but not limited to, magnetic resonance imaging, computed tomography scanning, and the like. In this manner, cancer cells are differentially labeled.

The detection methods can be provided as part of a kit. Thus, the invention further provides kits for detecting the presence of a MKPX, PTP4A1, PTPN7, FEM-2, DKFZP566K0524 or FLJ20313 mRNA, and/or a polypeptide encoded thereby, in a biological sample. Procedures using these kits can be performed by clinical laboratories, experimental laboratories, medical practitioners, or private Individuals. The kits of the invention for detecting a polypeptide comprise a moiety that specifically binds the polypeptide, which may be a specific antibody. The kits of the invention for detecting a nucleic acid comprise a moiety that specifically hybridizes to such a nucleic acid. The kit may optionally provide additional components that are useful in the procedure, including, but not limited to, buffers, developing reagents, labels, reacting surfaces, means for detection, control samples, standards, instructions, and interpretive information.

### Samples for Analysis

Sample of interest include tumor tissue, e.g. excisions, biopsies, blood samples where the tumoris metastatic, etc. Of particular interest are solid tumors, e.g. carcinomas, and include, without limitation, tumors of the liver and colon. Liver cancers of interest include hepatocellular carcinoma (primary liver cancer). Also called hepatoma, this Is the most common form of primary liver cancer. Chronic infection with hepatitis B and C Increases the risk of developing this type of cancer. Other causes include cancer-causing substances, alcoholism, and chronic liver cirrhosis. Other liver cancers of interest for analysis by the subject methods include hepatocellular adenoma, which are benign tumors occurring most often in women of childbearing age; hemangioma, which are a type of benign tumor comprising a mass of abnormal blood vessels, cholangiocarcinoma, which originates in the lining of the bile channels in the liver or in the bile ducts; hepatoblastoma, which Is common in infants and children; angiosarcoma, which is a rare cancer that originates in the blood vessels of the liver; and bile duct carcinoma and liver cysts. Cancers originating in the lung, breast, colon, pancreas and stomach and blood cells commonly are found in the liver after they become metastatic.

Also of interest are colon cancers. Types of polyps of the colon and rectum include polyps, which are any mass of tissue that arises from the bowel wall and protrudes into the lumen. Polyps may be sessile or pedunculated and vary considerably in size. Such lesions are classified histologically as tubular adenomas, tubulovillous adenomas (villoglandular polyps), villous (papillary) adenomas (with or without adenocarcinoma), hyperplasuc polyps, hamartomas, juvenile polyps, polypoid carcinomas, pseudopolyps, lipomas, lelomyomas, or other rarer tumors.

### Screening Methods

Target Screening. Reagents specific for MKPX, PTP4A1, PTPN7, FEM-2, DKFZP566K0524 or FLJ20313 are used to Identify targets of the encoded protein in tumor cells. For example, one of the nucleic acid coding sequences may be Introduced into a tumor cell using an inducible expression system. Suitable positive and negative controls are included. Transient transfection assays, e.g. using adenovirus vectors, may be performed. The cell system allows a comparison of the pattern of gene expression in transformed cells with or without expression of the phosphatase. Alternatively, phosphorylation patterns after induction of expression are examined. Gene expression of putative target genes may be monitored by Northern blot or by probing microarrays of candidate genes with the test sample and a negative control where gene expression of the phosphatase is not induced. Patterns of phosphorylation may be monitored by incubation of the cells or lysate with labeled phosphate, followed by 1 or 2 dimensional protein gel analysis, and identification of the targets by MALDI, micro-sequencing, Western blot analysis, etc., as known in the art.

Some of the potential target genes of the MKPX, PTP4A1, PTPN7, FEM-2, DKFZP566K0524 or FLJ20313 phosphatases Identified by this method will be secondary or tertiary in a complex cascade of gene expression or signaling. To identify primary targets of the subject phosphatase activation, expression or phosphorylation will be examined early after Induction of expression (within 1-2 hours) or after blocking later steps in the cascade with cycloheximide.

Target genes or proteins identified by this method may be analyzed for expression in primary patient samples as well. The data for the MKPX, PTP4A1, PTPN7, FEM-2, DKFZP566K0524 or FLJ20313 and target gene expression may be analyzed using statistical analysis to establish a correlation.

Compound Screening. The availability of a number of components in signaling pathways allows In vitro reconstruction of the pathway, and/or assessent of phosphatase action on targets. Two or more of the components may be combined in vitro, and the behavior assessed in terms of activation of transcription of specific target sequences; modification of protein components, e.g. proteolytic processing, phosphorylation, methylation, etc.; ability of different protein components to bind to each other etc. The components may be modified by sequence deletion, substitution, etc. to determine the functional role of specific domains.

Compound screening may be performed using an in vitro model, a genetically altered cell or animal, or purified MKPX, PTP4A1, PTPN7, FEM-2, DKFZP566K0524 or FLJ20313 protein. One can identify ligands or substrates that bind to, modulate or mimic the action of the encoded polypeptide. Areas of investigation include the development of treatments for hyper-proliferative disorders, e.g. cancer, restenosis, osteoarthritis, metastasis, etc.

The polypeptides include those encoded by SEQ ID NOS:1, 3, 5, 7, 9 or 11, as well as nucleic acids that, by virtue of the degeneracy of the genetic code, are not identical In sequence to the disclosed nucleic acids, and variants thereof. Variant polypeptides can include amino acid (aa) substitutions, additions or deletions. The amino acid substitutions can be conservative amino add substitutions or substitutions to eliminate non-essential amino acids, such as to alter a glycosylation site, a phosphorylation site or an acetylation site, or to minimize misfolding by substitution or deletion of one or more cysteine residues that are not necessary for function. Variants can be designed so as to retain or have enhanced biological activity of a particular region of the protein (e.g., a functional domain and/or, where the polypeptide Is a member of a protein family, a region associated with a consensus sequence). Variants also include fragments of the polypeptides disclosed herein, particularly biologically active fragments and/or fragments corresponding to functional domains. Fragments of interest will typically be at least about 10 aa to at least about 15 aa in length, usually at least about 50 aa In length, and can be as long as 300 aa in length or longer, but will usually not exceed about 500 aa In length, where the fragment will have a contiguous stretch of amino acids that Is identical to a polypeptide encoded by SEQ ID NOS:2, 4, 6, 8, 10 or 12, or a homolog thereof.

Transgenic animals or cells derived therefrom are also used in compound screening. Transgenic animals may be made through homologous recombination, where the normal locus corresponding to SEQ ID NOS:1, 3, 5, 7, 9 or 11 is altered. Alternatively, a nucleic acid construct is randomly integrated into the genome. Vectors for stable Integration include plasmids, retroviruses and other animal viruses, YACs, and the like. A series of small deletions and/or substitutions may be made In the coding sequence to determine the role of different exons in phosphatase activity, oncogenesis, signal transduction, etc. Of interest Is the use of SEQ ID NOS:1, 3, 5, 7, 9 or 11 to construct transgenic animal models for cancer, where expression of the corresponding phosphatase is specifically reduced or absent. Specific constructs of interest include antisense sequences that block expression of the targeted gene and expression of dominant negative mutations. A detectable marker, such as lac Z may be introduced into the locus of interest, where up-regulation of expression will result in an easily detected change in phenotype. One may also provide for expression of the target gene or variants thereof in cells or tissues where it Is not normally expressed or at abnormal times of development. By providing expression of the target protein in cells in which it is not normally produced, one can induce changes in cell behavior, e.g. In the control of cell growth and tumorigenesis.

Compound screening identifies agents that modulate function of MKPX, PTP4A1, PTPN7, FEM-2, DKFZP566K0524 or FLJ20313. Agents that mimic its function are predicted to activate the process of cell division and growth. Conversely, agents that Inhibit function may inhibit transformation. Of particular Interest are screening assays for agents that have a low toxicity for human cells. A wide variety of assays may be used for this purpose, including labeled in vitro protein-protein binding assays, electrophoretic mobility shift assays, immunoassays for protein binding, and the like. Knowledge of the 3-dimensional structure of the encoded protein, derived from crystallization of purified recombinant protein, could lead to the rational design of small drugs that specifically Inhibit activity. These drugs may be directed at specific domains, e.g. the phosphatase catalytic domain, the regulatory domain, the auto-inhibitory domain, etc.

The term “agent” as used herein describes any molecule, e.g. protein or pharmaceutical, with the capability of altering or mimicking the physiological function of MKPX, PTP4A1, PTPN7, FEM-2, DKFZP566K0524 or FLJ20313. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Where the screening assay Is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be Included In the screening assay. These include reagents like salts, neutral proteins, e.g. albumin, detergents, etc. that are used to facilitate optimal protein-protein binding and/or reduce non-specific or background interactions. Reagents that Improve the efficiency of the assay, such as protease inhibitors, nuclease Inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient

Other assays of interest detect agents that mimic the function of MKPX, PTP4A1, PTPN7, FEM-2, DKFZP566K0524 or FLJ20313. For example, an expression construct comprising the gene may be introduced into a cell line under conditions that allow expression. The level of phosphatase activity is determined by a functional assay, for example detection of protein phosphorylation. Alternatively, candidate agents are added to a cell that lacks MKPX, PTP4A1, PTPN7, FEM-2, DKFZP566K0524 or FLJ20313, and screened for the ability to reproduce the activity in a functional assay.

The compounds having the desired pharmacological activity may be administered in a physiologically acceptable carrier to a host for treatment of cancer, etc. The compounds may also be used to enhance function in wound healing, cell growth, etc. The inhibitory agents may be administered in a variety of ways, orally, topically, parenterally e.g. subcutaneously, intraperitoneally, by viral infection, intravascularly, etc. Depending upon the manner of introduction, the compounds may be formulated in a variety of ways. The concentration of therapeutically active compound in the formulation may vary from about 0.1-10 wt %.

Formulations. The compounds of this invention can be incorporated into a variety of formulations for therapeutic administration. Particularly, agents that modulate MKPX, PTP4A1, PTPN7, FEM-2, DKFZP566K0524 or FLJ20313 activity are formulated for administration to patients for the treatment of cells where the target activity Is undesirably high or low, e.g. to reduce the level of activity in cancer cells. More particularly, the compounds of the present Invention can be formulated Into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, Injections, inhalants, gels, microspheres, and aerosols. As such, administration of the compounds can be achieved in various ways, including oral, buccal, rectal, parenteral, intraperitoneal, intradermal, transdermal, intra-tracheal, etc., administration. The agent may be systemic after administration or may be localized by the use of an implant that acts to retain the active dose at the site of implantation.

In pharmaceutical dosage forms, the compounds may be administered in the form of their pharmaceutically acceptable salts, or they may also be used alone or In appropriate association, as well as in combination with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acaeia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier.

Implants for sustained release formulations are well-known in the art. Implants are formulated as microspheres, slabs, etc. with biodegradable or non-biodegradable polymers. For example, polymers of lactic add and/or glycolic acid form an erodible polymer that is well-tolerated by the host. The implant is placed in proximity to the site of disease, so that the local concentration of active agent is increased relative to the rest of the body.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Typical dosages for systemic administration range from 0.1 μg to 100 milligrams per kg weight of subject per administration. A typical dosage may be one tablet taken from two to six times daily, or one time-release capsule or tablet taken once a day and containing a proportionally higher content of active ingredient The time-release effect may be obtained by capsule materials that dissolve at different pH values, by capsules that release slowly by osmotic pressure, or by any other known means of controlled release.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a giveri compound.

The use of liposomes as a delivery vehicle is one method of interest. The liposomes fuse with the cells of the target site and deliver the contents of the lumen intracellularly. The liposomes are maintained in contact with the cells for sufficient time for fusion, using various means to maintain contact, such as isolation, binding agents, and the like. In one aspect of the invention, liposomes are designed to be aerosolized for pulmonary administration. Liposomes may be prepared with purified proteins or peptides that mediate fusion of membranes, such as Sendai virus or influenza virus, etc. The lipids may be any useful combination of known liposome forming lipids, including cationic lipids, such as phosphatidylcholine. The remaining lipid will normally be neutral lipids, such as cholesterol, phosphatidyl serine, phosphatidyl glycerol, and the like.

### Modulation of Enzyme Activity

Agents that block activity of MKPX, PTP4A1, PTPN7, FEM-2, DKFZP566K0524 or FLJ20313 provide a point of Intervention In an Important signaling pathway. Numerous agents are useful in reducing this activity, including agents that directly modulate expression as described above, e.g. expression vectors, antisense specific for the targeted phosphatase; and agents that act on the protein, e.g. specific antibodies and analogs thereof, small organic molecules that block catalytic activity, etc.

The genes, gene fragments, or the encoded protein or protein fragments are useful In therapy to treat disorders associated with defects in sequence or expression. From a therapeutic point of view, inhibiting activity has a therapeutic effect on a number of proliferative disorders, including inflammation, restenosis, and cancer. Inhibition is achieved in a number of ways. Antisense sequences may be administered to inhibit expression. Pseudo-substrate inhibitors, for example, a peptide that mimics a substrate for the phosphatase may be used to Inhibit activity. Other inhibitors are identified by screening for biological activity in a functional assay, e.g. in vitro or in vivo phosphatase activity.

Expression vectors may be used to Introduce the target gene into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced Into a variety of vectors, e.g. plasmid; retrovirus, e.g. lentivirus; adenovirus; and the like, where the vectors are able to transiently or stably be maintained In the cells, usually for a period of at least about one day, more usually for a period of at least about several days to several weeks.

The gene or protein may be Introduced into tissues or host cells by any number of routes, including viral Infection, microinjection, or fusion of vesicles. Jet injection may also be used for intramuscular administration, as described by Furth et al., Anal Biochem (1992) 205:365-368. The DNA may be coated onto gold microparticles, and delivered Intradermally by a particle bombardment device, or “gene gun” as described in the literature (see, for example, Tang et al., Nature (1992) 356:152-154), where gold micro-projectiles are coated with the protein or DNA, then bombarded into skin cells.

Antisense molecules can be used to down-regulate expression in cells. The antisense reagent may be antisense oligonucleotides (ODN), particularly synthetic ODN having chemical modifications from native nucleic adds, or nucleic acid constructs than express such antisense molecules as RNA. The antisense sequence Is complementary to the mRNA of the targeted gene, and Inhibits expression of the targeted gene products Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation Is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides In length, and not more than about 500, usually not more than about 50, more usually not more than about 35 nucleotides in length, where the length is governed by efficiency of inhibition, specificity, including absence of cross-reactivity, and the like, It has been found that short oligonucleotides, of from 7 to 8 bases in length, can be strong and selective Inhibitors of gene expression (see Wagner et al., Nature Biotechnology (1996) 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for Inhibition of expression of the target gene in vitro or In an animal model. A combination of sequences may also be used, where several regions of the mRNA sequence are selected for antisense complementation.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes In the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothloate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is Inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

### Therapeutic and Imaging Antibodies

Anti-phosphatase antibodies find for use therapeutic and imaging purposes. Such antibodies, which may be selected as described above, may be utilized without further modification to include a cytotoxic or Imaging moiety, or may be modified by conjugation to include such cytotoxic or imaging agents.

As used herein, “cytotoxic moiety” (C) simply means a moiety that inhibits cell growth or promotes cell death when proximate to or absorbed by the cell. Suitable cytotoxic moieties in this regard include radioactive isotopes (radionuclides), chemotoxic agents such as differentiation inducers and small chemotoxic drugs, toxin proteins, and derivatives thereof. As utilized herein, “imaging moiety” (I) means a moiety which can be utilized to increase contrast between a tumor and the surrounding healthy tissue In a visualization technique (e.g., radiography, positron-emission tomography, magnetic resonance imaging, direct or indirect visual inspection.) Thus, suitable imaging moieties include radiography moieties (e.g. heavy metals and radiation emitting moieties), positron emitting moieties, magnetic resonance contrast moieties, and optically visible moieties (e.g., fluorescent or visible-spectrum dyes, visible particles, etc.). It will be appreciated by one of ordinary skill that some overlap exists between what is a therapeutic moiety and what is an Imaging moiety. For instance ²¹²Pb and ²¹²Bi are both useful radioisotopes for therapeutic compositions, but are also electron-dense, and thus provide contrast for X-ray radiographic Imaging techniques, and can also be utilized in scintillation Imaging techniques.

In general, therapeutic or imaging agents may be conjugated to the anti-phosphatase moiety by any suitable technique, with appropriate consideration of the need for pharmokinetic stability and reduced overall toxicity to the patient. A therapeutic agent may be coupled to a suitable antibody moiety either directly or indirectly (e.g. via a linker group). A direct reaction between an agent and an antibody is possible when each possesses a functional group capable of reacting with the other. For example, a nucleophilic group, such as an amino or sulfhydryl group, may be capable of reacting with a carbonyl-containing group, such as an anhydride or an acid halide, or with an alkyl group containing a good leaving group (e.g., a halide). Alternatively, a suitable chemical linker group may be used. A linker group can function as a spacer to distance an antibody from an agent In order to avoid interference with binding capabilities. A linker group can also serve to increase the chemical reactivity of a substituent on a moiety or an antibody, and thus increase the coupling efficiency. An increase in chemical reactivity may also facilitate the use of moieties, or functional groups on moieties, which otherwise would not be possible.

Suitable linkage chemistries Include maleimidyl linkers and alkyl halide linkers (which react with a sulfhydryl on the antibody moiety) and succinimidyl linkers (which react with a primary amine on the antibody moiety). Several primary amine and sulfhydryl groups are present on immunoglobulins, and additional groups may be designed into recombinant Immunoglobulin molecules. It will be evident to those skilled in the art that a variety of bifunctional or polyfunctional reagents, both homo- and heterofunctional (such as those described in the catalog of the Pierce Chemical Co., Rockford, Ill.), may be employed as a linker group. Coupling may be effected, for example, through amino groups, carboxyl groups, sulfhydryl groups or oxidized carbohydrate residues. There are numerous references describing such methodology, e.g., U.S. Pat. No. 4,671,958. As an alternative coupling method, cytotoxic or imaging moieties may be coupled to the antibody moiety through an oxidized carbohydrate group at a glycosylation site, as described in U.S. Pat. Nos. 5,057,313 and 5,156,840. Yet another alternative method of coupling the antibody moiety to the cytotoxic or imaging moiety is by the use of a non-covalent binding pair, such as streptavidin/biotin, or avidin/biotin. In these embodiments, one member of the pair is covalently coupled to the antibody moiety and the other member of the binding pair Is covalently coupled to the cytotoxic or imaging moiety.

Where a cytotoxic moiety is more potent when free from the antibody portion of the immunoconjugates of the present invention, it may be desirable to use a linker group that Is cleavable during or upon internalization into a cell, or that Is gradually cleavable over time in the extracellular environment. A number of different cleavable linker groups have been described. The mechanisms for the Intracellular release of a cytotoxic moiety agent from these linker groups include cleavage by reduction of a disulfide bond (e.g., U.S. Pat. No. 4,489,710), by Irradiation of a photolabile bond (e.g., U.S. Pat. No. 4,625,014), by hydrolysis of derivatized amino acid side chains (e.g., U.S. Pat. No. 4,638,045), by serum complement-mediated hydrolysis (e.g., U.S. Pat. No. 4,671,958), and acid-catalyzed hydrolysis (e.g., U.S. Pat. No. 4,569,789).

It may be desirable to couple more than one cytotoxic and/or Imaging moiety to an antibody. By poly-derivatizing the antibody, several cytotoxic strategies may be simultaneously implemented, an antibody may be made useful as a contrasting agent for several visualization techniques, or a therapeutic antibody may be labeled for tracking by a visualization technique. In one embodiment, multiple molecules of an Imaging or cytotoxic moiety are coupled to one antibody molecule. In another embodiment, more than one type of moiety may be coupled to one antibody. Regardless of the particular embodiment, immunoconjugates with more than one moiety may be prepared in a variety of ways. For example, more than one moiety may be coupled directly to an antibody molecule, or linkers which provide multiple sites for attachment (e.g., dendrimers) can be used. Alternatively, a carrier with the capacity to hold more than one cytotoxic or imaging moiety can be used.

A carrier may bear the agents in a variety of ways, including covalent bonding either directly or via a linker group, and non-covalent associations. Suitable covalent-bond carriers Include proteins such as albumins (e.g., U.S. Pat. No. 4,507,234), peptides, and polysaccharides such as aminodextran (e.g., U.S. Pat. No. 4,699,784), each of which have multiple sites for the attachment of moieties. A carrier may also bear an agent by non-covalent associations, such as non-covalent bonding or by encapsulation, such as within a liposome vesicle (e.g., U.S. Pat. Nos. 4,429,008 and 4,873,088). Encapsulation carriers are especially useful for imaging moiety conjugation to antibody moieties for use in the invention, as a sufficient amount of the imaging moiety (dye, magnetic resonance contrast reagent, etc.) for detection may be more easily associated with the antibody moiety. In addition, encapsulation carriers are also useful in chemotoxic therapeutic embodiments, as they can allow the therapeutic compositions to gradually release a chemotoxic moiety over time while concentrating it in the vicinity of the tumor cells.

Carriers and linkers specific for radionuclide agents (both for use as cytotoxic moieties or positron-emission imaging moieties) include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis. Such chelation carriers are also useful for magnetic spin contrast ions for use In magnetic resonance imaging tumor visualization methods, and for the chelation of heavy metal Ions for use in radiographic visualization methods.

Preferred radionuclides for use as cytotoxic moieties are radionulcides which are suitable for pharmacological administration. Such radionuclides Include ¹²³I, ¹²⁵I, ¹³¹I, ⁹⁰Y, ²¹¹At, ⁶⁷Cu, ¹⁸⁸Re, ¹⁸⁸Re, ²¹²Pb, and ²¹²Bi. Iodine and astatine Isotopes are more preferred radionuclides for use in the therapeutic compositions of the present Invention, as a large body of literature has been accumulated regarding their use. ¹³¹I is particularly preferred, as are other 1-radiation emitting nuclides, which have an effective range of several millimeters. ¹²³I, ¹²⁵I, ¹³¹I, or ²¹¹At may be conjugated to antibody moieties for use in the compositions and methods utilizing any of several known conjugation reagents, Including Iodogen, N-succinimidyl 3-[²¹¹At]astatobenzoate, N-succinimidyl 3-[¹³¹I]iodobenzoate (SIB), and, N-succinimidyl 5-[³¹¹I]iodob-3-pyridinecarboxylate (SIPC). Any iodine isotope may be utilized in the recited Iodo-reagents. For example, a suitable antibody for use in the present invention may be easily made by coupling an Fab fragment of the BD Transduction Labs R20720 anti-SEQ ID NOS:2, 4, 6, 8, 10 or 12 MAb with ¹³¹I Iodogen according to the manufacturer's instructions. Other radionuclides may be conjugated to anti-SEQ ID NOS:2, 4, 6, 8, 10 or 12 antibody moieties by suitable chelation agents known to those of skill in the nuclear medicine arts.

Preferred chemotoxic agents include small-molecule drugs such as methotrexate, and pyrimidine and purine analogs. Preferred chemotoxin differentiation inducers include phorbol esters and butyric acid. Chemotoxic moieties may be directly conjugated to the antibody moiety via a chemical linker, or may encapsulated in a carrier, which is in turn coupled to the antibody moiety.

Preferred toxin proteins for use as cytotoxic moieties include ricin, abrin, diphtheria toxin, cholera toxin, gelonin, Pseudomonas exotoxin, Shigella toxin, pokeweed antiviral protein, and other toxin proteins known in the medicinal biochemistry arts. As these toxin agents may elicit undesirable Immune responses in the patient, especially if Injected intravascularly, it is preferred that they be encapsulated in a carrier for coupling to the antibody moiety.

Preferred radiographic moieties for use as imaging moieties in the present invention include compounds and chelates with relatively large atoms, such as gold, iridium, technetium, barium, thallium, iodine, and their isotopes. It is preferred that less toxic radiographic imaging moieties, such as iodine or iodine isotopes, be utilized in the compositions and methods of the invention. Examples of such compositions which may be utilized for x-ray radiography are described in U.S. Pat. No. 5,709,846, incorporated fully herein by reference. Such moieties may be conjugated to the anti-SEQ ID NOS:2, 4, 6, 8, 10 or 12 antibody moiety through an acceptable chemical linker or chelation carrier. Positron emitting moieties for use in the present invention include ¹⁸F, which can be easily conjugated by a fluorination reaction with the antibody moiety according to the method described in U.S. Pat. No. 6,187,284.

Preferred magnetic resonance contrast moieties include chelates of chromium(II), manganese(II), iron(II), nickel(II), copper(II), praseodymium(II), neodymium(III), samarium(III) and ytterbium(III) ion. Because of their very strong magnetic moment, the gadolinium(III), terbium(III), dysprosium(III), holmium(III), erbium(III), and iron(III) ions are especially preferred. Examples of such chelates, suitable for magnetic resonance spin imaging, are described in U.S. Pat. No. 5,733,522, Incorporated fully herein by reference. Nuclear spin contrast chelates may be conjugated to the antibody moieties through a suitable chemical linker.

Optically visible moieties for use as imaging moieties include fluorescent dyes, or visible-spectrum dyes, visible particles, and other visible labeling moieties. Fluorescent dyes such as fluorescein, coumarin, rhodamine, bodipy Texas red, and cyanine dyes, are useful when sufficient excitation energy can be provided to the site to be inspected visually. Endoscopic visualization procedures may be more compatible with the use of such labels. For many procedures where imaging agents are useful, such as during an operation to resect a brain tumor, visible spectrum dyes are preferred. Acceptable dyes Include FDA-approved food dyes and colors, which are non-toxic, although pharmaceutically acceptable dyes that have been approved for internal administration are preferred. In some embodiments, such dyes are encapsulated in carrier moieties, which are In turn conjugated to the antibody. Alternatively, visible particles, such as colloidal gold particles or latex particles, may be coupled to the antibody moiety via a suitable chemical linker.

For administration, the antibody-therapeutic or antibody-imaging agent will generally be mixed, prior to administration, with a non-toxic, pharmaceutically acceptable carrier substance. Usually, this will be an aqueous solution, such as normal saline or phosphate-buffered saline (PBS), Ringer's solution, lactate-Ringer's solution, or any isotonic physiologically acceptable solution for administration by the chosen means. Preferably, the solution is sterile and pyrogen-free, and is manufactured and packaged under current Good Manufacturing Processes (GMP), as approved by the FDA or HPB. The clinician of ordinary skill is familiar with appropriate ranges for pH, tonicity, and additives or preservatives when formulating pharmaceutical compositions for administration by intravascular injection, intrathecal injection, injection into the cerebro-spinal fluid, direct injection into the tumor, or by other routes. In addition to additives for adjusting pH or tonicity, the antibody-therapeutics and antibody-imaging agents may be stabilized against aggregation and polymerization with amino acids and non-ionic detergents, polysorbate, and polyethylene glycol. Optionally, additional stabilizers may Include various physiologically-acceptable carbohydrates and salts. Also, polyvinylpyrrolidone may be added In addition to the amino acid. Suitable therapeutic immunoglobulin solutions which are stabilized for storage and administration to humans are described in U.S. Pat. No. 5,945,098, incorporated fully herein by reference. Other agents, such as human serum albumin (HSA), may be added to the therapeutic or imaging composition to stabilize the antibody conjugates. Antibodies coupled to cytotoxic moieties will recognize their targets within the body, where the cytotoxic moiety is brought in contact to or in dose proximity to the a tumor, whereupon the cytotoxic moiety interferes with the tumor and reduces it's growth, reduces is size, prevents metastasis, or otherwise kills the cells in the tumor. Antibodies coupled to imaging moieties will recognize their targets within the body, whereupon their targets can be visualized using suitable methods described above, as Is appropriate for the imaging moiety used.

### EXAMPLES

The following examples are put forth so as to provide those of ordinary skill in the art with a complete disclosure and description of how to make and use the present Invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein Incorporated by reference as if each Individual publication or patent application were specifically and individually indicated to be incorporated by reference.

The present invention has been described in terms of particular embodiments found or proposed by the present Inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Identification of Phosphatase Sequences

The Genbank database was searched for ESTs showing similarity to known phosphatase domain-related proteins using the “basic local alignment search tool” program, TBLASTN, with default settings. Human ESTs identified as having similarity to these known phosphatase domains (defined as p<0.0001) were used In a BLASTN and BLASTX screen of the Genbank non-redundant (NR) database.

ESTs that had top human hits with >95% identity over 100 amino acids were discarded. The remaining BLASTN and BLASTX outputs for each EST were examined manually, i.e., ESTs were removed from the analysis if the inventors determined that the variation from the known phosphatase domain-related probe sequence was a result of poor database sequence. Poor database sequence was usually identified as a number of ‘N’ nucleotides in the database sequence for a BLASTN search and as a base deletion or insertion in the database sequence, resulting in a peptide frameshift, for a BLASTX output. ESTs for which the highest scoring match was to non-phosphatase domain-related sequences were also discarded at this stage.

Using widely known algorithms, e.g. “Smith/Waterman”, “FastA”, “FastP”, “Needleman/Wunsch”, “Blast”, “PSIBlast,” homology of the subject nucleic acid to other known nucleic adds was determined. A “Local FastP Search” algorithm was performed in order to determine the homology of the subject nucleic add invention to known sequences. Then, a ktup value, typically ranging from 1 to 3 and a segment length value, typically ranging from 20 to 200, were selected as parameters. Next, an array of position for the probe sequence was constructed In which the cells of the array contain a list of positions of that substring of length ktup. For each subsequence in the position array, the target sequence was matched and augmented the score array cell corresponding to the diagonal defined by the target position and the probe subsequence position. A list was then generated and sorted by score and report. The criterion for perfect matches and for mismatches was based on the statistics properties of that algorithm and that database, typically the values were: 98% or more match over 200 nucleotides would constitute a match; and any mismatch in 20 nucleotides would constitute a mismatch.

Analysis of the BLASTN and BLASTX outputs identified an EST sequence from an IMAGE clone that had potential for being associated with a sequence encoding a phosphatase domain-related protein, e.g., the sequence had homology, but not identity, to known phosphatase domain-related proteins.

After identification of phosphatase ESTs, the clones were added to Kinetek's clone bank for analysis of gene expression In tumor samples. Gene expression work involved construction of unigene clusters, which are represented by entries In the “pks” database. A list of accession numbers for members of the clusters were assigned. Subtraction of the clusters already present in the clone bank from the dusters recently added left a list of clusters that had not been previously represented in Kinetek's clone bank. For each of the clusters, a random selection of an EST IMAGE accession numbers were chosen to represent the clusters. For each of the clusters which did not have an EST IMAGE clone, generation of a report so that clone ordering or construction could be implemented was performed on a case by case basis. A list of accession numbers which were not in dusters was constructed and a report was generated.

The identified IMAGE clones were sequenced using standard ABI dye-primer and dye-terminator chemistry on a 377 automatic DNA sequencer.

### Example 2

### Expression Analysis of MKPX. PTP4A1. PTPN7. FEM-2. DKFZP566K0524 and FLJ20313

The expression of MKPX, PTP4A1, PTPN7, FEM-2, DKFZP566K0524 and FLJ20313 was determined by dot blot analysis, and the proteins were found to be upregulated in several tumor samples.

Dot blot preparation. Total RNA was purified from clinical cancer and control samples taken from the same patient. Samples were used from colon tumors. Using reverse transcriptase, cDNAs were synthesized from these RNAs. Radiolabeled cDNA was synthesized using Strip-EZ™ kit (Ambion, Austin, Tex.) according to the manufacturer's instructions. These labeled, amplified cDNAs were then used as a probe, to hybridize to human phosphatase arrays comprising human MKPX, PTP4A1, PTPN7, FEM-2, DKFZP566K0524 and FLJ20313 sequences. The amount of radiolabeled probe hybridized to each arrayed EST clone was detected using phosphorimaging. The expression of these genes was substantially upregulated In at least one of the tumor tissues tested. Samples are taken from the colon, prostate, breast, kidney, uterine, kidney, stomach, bladder, leukemia, cervical tumors, using dot blots or RT-PCR, expression of MKPX, PTP4A1, PTPN7, FEM-2, DKFZP566K0524 and FLJ20313 is examined.

### Example 3

### Antisense Regulation of MKPX. PTP4A1. PTPN7. FEM-2. DKFZP566K0524 or FLJ20313 Expression

Additional functional information on MKPX, PTP4A1, PTPN7, FEM-2, DKFZP566K0524 or FLJ20313 is generated using antisense knockout technology. MKPX, PTP4A1, PTPN7, FEM-2, DKFZP566K0524 or FLJ20313 expression in cancerous cells is further analyzed to confirm the role and function of the gene product in tumorgenesis, e.g., in promoting a metastatic phenotype.

A number of different oligonucleotides complementary to MKPX, PTP4A1, PTPN7, FEM-2, DKFZP566K0524 or FLJ20313 mRNA are designed as potential antisense oligonucleotides, and tested for their ability to suppress expression of one of the peptides of the Invention. The ability of each designed antisense oligonucleotide to inhibit gene expression is tested through transfection into SW620 colon colorectal carcinoma cells, or cells from any other cell lines such as A546 (Lung carcinoma), B16-F1 (Melanoma), DLD-1 (Colon carcinoma), LS-180 (Colon carcinoma), PC3 (Prostate carcinoma), U87 (Glioma), MCF-7 (Mammary carcinoma), Huvec (normal human endothelial), Hs-27 (normal lung fibroblast) and MCF-10a (Mammary epithelial). For each transfection mixture, a carrier molecule, preferably a lipitoid or cholesteroid, is prepared to a working concentration of 0.5 mM in water, sonicated to yield a uniform solution, and filtered through a 0.45 μm PVDF membrane. The antisense or control oligonucleotide Is then prepared to a working concentration of 100 μM in sterile Millipore water. The oligonucleotide is further diluted in OptiMEM™ (Gibco/BRL), in a microfuge tube, to 2 μM, or approximately 20 μg oligo/ml of OptiMEM™. In a separate microfuge tube, lipitoid or cholesteroid, typically in the amount of about 1.5-2 nmol lipitoid/μg antisense oligonucleotide, is diluted into the same volume of OptiMEM™ used to dilute the oligonucleotide. The diluted antisense oligonucleotide Is immediately added to the diluted lipitoid and mixed by pipetting up and down. Oligonucleotide Is added to the cells to a final concentration of 30 nM. The level of target mRNA in the transfected cells is quantitated in the cancer cell lines using the Roche LightCycler™ real-time PCR machine. Values for the target mRNA is normalized versus an internal control (e.g., beta-actin).

The antisense oligonucleotides are Introduced Into a test cell and the effect upon MKPX, PTP4A1, PTPN7, FEM-2, DKFZP566K0524 or FLJ20313 expression, as well as the effect upon induction of the cancerous phenotype, is examined as described below.

### Example 4

### Effects of MKPX. PTP4A1. PTPN7. FEM-2. DKFZP566K0524 or FLJ20313 Antisense Polynucleotides on Cell Proliferation

The effect of MKPX, PTP4A1, PTPN7, FEM-2, DKFZP566K0524 or FLJ20313 antisense polynucleotides on proliferation is assessed in the cancer cell lines listed above. Transfection Is carried out as described above In Example 4, except the final concentration of oligonucleotide for all experiments Is 300 nM, and the final ratio of oligo to delivery vehicle for all experiments is 1.5 nmol lipitoid/μg oligonucleotide. Cells are transfected overnight at 37° C. and the transfection mixture is replaced with fresh medium the next morning. Proliferation is measured visually and the effects of antisense polynucleotides on cell proliferation are determined.

### Example 5

### Effects of MKPX, PTP4A1, PTPN7, FEM-2, DKFZP566K0524 or FLJ20313 Antisense Polynucleotides on Colony Formation

The effect of MKPX, PTP4A1, PTPN7, FEM-2, DKFZP566K0524 or FLJ20313 antisense polynucleotides on colony formation is tested in a soft agar assay. Soft agar assays are conducted by first establishing a bottom layer of 2 ml of 0.6% agar in media plated fresh within a few hours of layering on the cells. The cell layer is formed on the bottom layer by removing cells transfected as described above from plates using 0.05% trypsin and washing twice in media. The cells are counted in a Coulter counter, and resuspended to 106 per ml in media. 10 μl aliquots are placed with media In 96-well plates, or diluted further for soft agar assay. Cells are plated in 0.4% agar in duplicate wells above 0.6% agar bottom layer. After the cell layer agar solidifies, 2 ml of media Is dribbled on top and antisense or reverse control oligo is added without delivery vehicles. Colonies are formed in 10 days to 3 weeks. Fields of colonies are counted by eye and the effects of antisense polynucleotides on colony formation can be determined.

### Example 6

### Induction of Cell Death Upon Depletion of MKPX, PTP4A1, PTPN7, FEM-2, DKFZP566K0524 or FLJ20313

Cells are transfected as described for proliferation assays. Each day, cytotoxicity is monitored by measuring the amount of LDH enzyme released in the medium due to membrane damage. The activity of LDH Is measured using the Cytotoxicity Detection Kit from Roche Molecular Biochemicals. The data is provided as a ratio of LDH released in the medium vs. the total LDH present in the well at the same time point and treatment (rLDH/tLDH).

### Example 7

### Assay for Agents that Modulate MKPX, PTP4A1, PTPN7, FEM-2, DKFZP566K0524 or FLJ20313 Activity

MKPX, PTP4A1, PTPN7, FEM-2, DKFZP566K0524 or FLJ20313 is expressed as a 6× His tag fusion protein using the baculovirus system, purified using affinity chromatography, and phosphatase assays are performed as described In Ausubel et al 1999 (Short protocols in molecular biology, John Wiley and Sons, NY).

Agents modulating MKPX, PTP4A1, PTPN7, FEM-2, DKFZP566K0524 or FLJ20313 activity can be identified by comparing the activity of one of the phosphatases in the presence of a candidate agent to the activity of the same phosphatase in the absence of a candidate agent.

1. A method of screening for biologically active agents that modulate a cancer associated phosphatase function, the method comprising:
combining a candidate biologically active agent with any one of:
(a) a polypeptide encoded by SEQ ID NOS:1, 3, 5, 7, 9 or 11; or having the amino acid sequence set forth in SEQ ID NOS:2, 4, 6, 8, 10 or 12;
(b) a cell comprising a nucleic acid encoding a polypeptide encoded by SEQ ID NOS: 1, 3, 5, 7, 9 or 11; or
(c) a non-human transgenic animal model for cancer associated phosphatase gene function comprising one of: (i) a knockout of a gene corresponding to SEQ ID NOS: 1, 3, 5, 7, 9 or 11; (ii) an exogenous and stably transmitted mammalian gene sequence comprising polypeptide encoded by SEQ ID NOS: 1, 3, 5, 7, 9 or 11; and determining the effect of said agent on phosphatase function.

2. A method for the diagnosis of cancer, the method comprising:
determining the upregulation of expression in SEQ ID NOS: 1, 3, 5, 7, 9 or 11 in said cancer.

3-6. (canceled)

7. A method for inhibiting the growth of a cancer cell, the method comprising:
downregulating activity of the polypeptide encoded by SEQ ID NOS: 1, 3, 5, 7, 9 or 11; or
having the amino acid sequence set forth in SEQ ID NOS:2, 4, 6, 8, 10 or 12; in said cancer cell.

8-11. (canceled)

12. A method of screening for targets of a cancer associated phosphatase, wherein said targets are associated with signal transduction in cancer cells, the method comprising:
comparing the pattern of gene expression or protein phosphorylation in a normal cell, and in a tumor cell characterized by up-regulation of SEQ ID NOS: 1, 3, 5, 7, 9 or 11.

13-14. (canceled)

15. The method according to claim 12 or claim 11, wherein said signal transduction involves activation MKPX, PTP4A1, PTPN7, FEM-2, DKFZP566K0524 or FLJ20313.

16. An isolated nucleic acid comprising the sequence set forth in SEQ ID NOS: 1, 3, 5, 7, 9 or 11.

17. A method to treat a tumor comprising administering a therapeutic amount of a composition comprising:
a compound of the general formula general formula α(P_{z}), wherein α(P_{z}) is one or more moieties which specifically binds to a human protein MKPX, PTP4A1, PTPN7, FEM-2, DKFZP566K0524 or FLJ20313, wherein the binding of α(P_{z}) alters the function of the human protein α(P_{z}) or wherein α(P_{z}) comprises one or more cytotoxic moieties;
and a pharmaceutically acceptable carrier.

18-27. (canceled)

28. A compound for the treatment of a tumor of the general formula α(P_{z}), wherein α(P_{z}) is one or more moieties which specifically binds to human MKPX, PTP4A1, PTPN7, FEM-2, DKFZP566K0524 or FLJ20313 protein, and alters the function of the Protein or comprises one or more cytotoxic moieties.

29-42. (canceled)

43. A method for visualizing a tumor in a patient, the method comprising:
(a) administering to a patient an effective amount of a composition comprising:
a compound of the general formula α(P_{z})I, wherein α(P_{z}) is one or more moieties which specifically binds to a human MKPX, PTP4A1, PTPN7, FEM-2, DKFZP566K0524 or FLJ20313 protein, and I is one or more imaging moieties; and a pharmaceutically acceptable carrier; and (b) visualizing the imaging moieties of the compound.

44-59. (canceled)


SEQUENCE LISTING (12 sequences)

SEQ ID NO: 1 - DNA, 1520 bp, Homo sapiens
  misc_feature (0)...(0): MKPX polynucleotide
ggcacgaggccgagcctagtgcctcccacgcccggcggccgcgagccggggtccgcgagggcggagtggggcgcggcagccaggaacccgactacgaatcccagggtgcgggcgggcggagcgaggagggacgctgggcctgcccggtgcgcacgggggcggggaccggcaaggcgggaccatttcccggcataggctccggtgcccctgcccggctcccgccgggaagttctaggccgccgcacagaaagccctgccctccacgccgggtctctggagcgccctgggttgcccggccggtccctgccgctgacttgttgacactgcgagcactcagtccctcccgcgcgcctcctccccgcccgccccgccgctcctcctccctgtaacatgccatagtgcgcctgcgaccacacggccggggcgctagcgttcgccttcagccaccatggggaatgggatgaacaagatcctgcccggcctgtacatcggcaacttcaaagatgccagagacgcggaacaattgagcaagaacaaggtgacacatattctgtctgtccatgatagtgccaggcctatgttggagggagttaaatacctgtgcatcccagcagcggattcaccatctcaaaacctgacaagacatttcaaagaaagtattaaattcattcacgagtgccggctccgcggtgagagctgccttgtacactgcctggccggggtctccaggagcgtgacactggtgatcgcatacatcatgaccgtcactgactttggctgggaggatgccctgcacaccgtgcgtgctgggagatcctgtgccaaccccaacgtgggcttccagagacagctccaggagtttgagaagcatgaggtccatcagtatcggcagtggctgaaggaagaatatggagagagccctttgcaggatgcagaagaagccaaaaacattctggccgctccaggaattctgaagttctgggcctttctcagaagactgtaatgtacctgaagtttctgaaatattgcaaacccacagagtttaggctggtgctgccaaaaagaaaagcaacatagagtttaagtatccagtagtgatttgtaaacttgtttttcatttgaagctgaatatatacgtagtcatgtttatgttgagaactaaggatattctttagcaagagaaaatattttccccttatccccactgctgtggaggtttctgtacctcgcttggatgcctgtaaggatcccgggagccttgccgcactgccttgtgggtggcttggcgctcgtgattgcttcctgtgaacgcctcccaaggacgagcccagtgtagttgtgtggcgtgaactctgcccgtgtgttctcaaattccccagcttgggaaatagcccttggtgtgggttttatctctggtttgtgttctccgtggtggaattgaccgaaagctctatgttttcgttaataaagggcaacttagccaagtttaaaaaaaaaaaaaaaaaaaaaaaaaaaaaaaa

SEQ ID NO: 2 - protein, 184 aa, Homo sapiens
  UNSURE (0)...(0): MKPX polypeptide
MGNGMNKILPGLYIGNFKDARDAEQLSKNKVTHILSVHDSARPMLEGVKYLCIPAADSPSQNLTRHFKESIKFIHECRLRGESCLVHCLAGVSRSVTLVIAYIMTVTDFGWEDALHTVRAGRSCANPNVGFQRQLQEFEKHEVHQYRQWLKEEYGESPLQDAEEAKNILAAPGILKFWAFLRRL

SEQ ID NO: 3 - DNA, 2916 bp, Homo sapiens
  misc_feature (0)...(0): PTP4A1 polynucleotide
aagggcgcctcggcgcgtgtattggctccttcggctgcgggccggctcgcctacgcgctctgctccgagccgctcactgcatggtagagtctggtgcccccgccgccgcctgcatcgccgccaccgccgctccgccacgaccaccgccgcctccttgtcctgcagccaccgccaccgcctgtgtcgccgccgctcgggaccggctgtatgattaggccacaatcttcaatgagtaaacatattcctcaattctgtggtgttcttggtcacacatttatggagtttctgaagggcagtggagattactgccaggcacagcacgacctctatgcagacaagtgaactgtagaaactgattactgctccaccaagaagcccccataagagtggttatcctggacacagaagtgttgaaatccacagagcattttacaagagttctgacctggatggggtaaacctcagtgcacttcttttctgttggcctcagtattactggattgaagaattgctgcttcttgttaggaggttcatttcacttatcattacttacaacttcatactcaaagcactgagaatttcaagtggagtatattgaagtagacttcagtttctttggatcatttctgtattcaatttttttaattatttcataaccctattgagtgttttttaactaaattaacatggctcgaatgaaccgcccagctcctgtggaagtcacatacaagaacatgagatttcttattacacacaatccaaccaatgcgaccttaaacaaatttatagaggaacttaagaagtatggagttaccacaatagtaagagtatgtgaagcaacttatgacactactcttgtggagaaagaaggtatccatgttcttgattggccttttgatgatggtgcaccaccatccaaccagattgttgatgactggttaagtcttgtgaaaattaagtttcgtgaagaacctggttgttgtattgctgttcattgcgttgcaggccttgggagagctccagtacttgttgccctagcattaattgaaggtggaatgaaatacgaagatgcagtacaattcataagacaaaagcggcgtggagcttttaacagcaagcaacttctgtatttggagaagtatcgtcctaaaatgcggctgcgtttcaaagattccaacggtcatagaaacaactgttgcattcaataaaattggggtgcctaatgctactggaagtggaacttgagatagggcctaatttgttatacatattagccaacatgttggcttagtaagtctaatgaagcttccataggagtattgaaaggcagttttaccaggcctcaagctagacagatttggcaacctctgtatttgggttacagtcaacctatttggatacttggcaaaagattcttgctgtcagcatataaaatgtgcttgtcatttgtatcaattgacctttccccaaatcatgcagtattgagttatgacttgttaaatctattcccatgccagaatcttatcaatacataagaaatttaggaagattaggtgccaaaatacccagcacaatacttgtatatttttagtaccatacagaagtaaaatcccaggaactatgaacactagaccttatgtggtttattccttcagtcatttcaaacattgaaagtagggcctacatggttatttggctgctcactttatgtttacatctcccacattcataccaatatacgtcaggtttggttaaccattgatttttttttttttttaccaagtcttacagtgattattttacgtgtttccatgtatctcactttgtgctgtattaaaaaaacctccattttgaaaatctacgttgtacagaagcacatgtctttaatgtcttcagacaaaaaagccttacattaatttaatgtttgcactctgaggtgcaacttaacagggagggcctgagaaaagaatgggagggggctattaattatttttagcaaaatgttgcctttgtcttgtgcaaacatgtagaatatgctctttaatttagtaaaatatttttttaaaaggtagagatgctttgttattgtaatcataaacttcctgaaattcttgtaatttttttcccatacttatcagaagtgtgtttaccaacttattcttgtttgaaagtgtgattttttttttccttcccaacctctcttgcaaaaaaagaaatgggtttctgctaatgaattgagcagacatctaatattttatatgccttttggagctgggtaacttaatatttggatacttgacaatttgttttattatgtaattgataaaatggtgatgtgtattaatgttagttcaaccatatatttatactgtctggggatgtgtggttatagttctgtgggagaaataattttgtcagtgttcaccagcttgtaaaaacttagtgcgagagctgaaacatctaaataaataatgacatgcatttatcatcattgagattggtttgcttaaaattaacttattttgtagaagacaaaatgaattgcacttcacttaatgtgtgtcctcatctttttacaaataaatgaaggattataaatgatgtcagcattttagtaaacttatagacaaaatttgttagggtcattcatgaaaactttaatactaaaagcactttccattatatactttttaaaggtctagataattttgaaccaatttattattgtgtactgaggagaaataatgtatagtagaggacagccttggtttgtaaagctcagctccactagttcatggtttggtgcaacttctgagcctcagttctctcctttgcaaattaataattacatacctgcctagatttcggaaattaatctaaatattagtatctggctacatgatggccatgtcaagt

SEQ ID NO: 4 - protein, 173 aa, Homo sapiens
  UNSURE (0)...(0): PTP4A1 polypeptide sequence
MARMNRPAPVEVTYKNMRFLITHNPTNATLNKFIEELKKYGVTTIVRVCEATYDTTLVEKEGIHVLDWPFDDGAPPSNQIVDDWLSLVKIKFREEPGCCIAVHCVAGLGRAPVLVALALIEGGMKYEDAVQFIRQKRRGAFNSKQLLYLEKYRPKMRLRFKDSNGHRNNCCIQ

SEQ ID NO: 5 - DNA, 2759 bp, Homo sapiens
  misc_feature (0)...(0): PTPN7 polynucleotide sequence
ggcacgaggcaagaggcagcctgggggccacagctgcttcagcagacctcatggctgagtgagcctcccctgggcccagcaccccacctcagcatggtccaagccatggggggcgctccagagcacagccgttgaccttgtctttgggggcagccatgacccagcctccgcctgaaaaaacgccagccaagaagcatgtgcgactgcaggagaggcggggctccaatgtggctctgatgctggacgttcggtccctgggggccgtagaacccatctgctctgtgaacacaccccgggaggtcaccctacactttctgcgcactgctggacacccccttacccgctgggcccttcagcgccagccacccagccccaagcaactggaagaagaattcttgaagatcccttcaaactttgtcagccccgaagacctggacatccctggccacgcctccaaggaccgatacaagaccatcttgccaaatccccagagccgtgtctgtctaggccgggcacagagccaggaggacggagattacatcaatgccaactacatccgaggctatgacgggaaggagaaggtctacattgccacccagggccccatgcccaacactgtgtcggacttctgggagatggtgtggcaagaggaagtgtccctcattgtcatgctcactcagctccgagagggcaaggagaaatgtgtccactactggcccacagaagaggaaacctatggacccttccagatccgcatccaggacatgaaagagtgcccagaatacactgtgcggcacgtcaccatccagtaccaggaagagcgccggtcagtaaagcacatcctcttttcggcctggccagaccatcagacaccagaatcagctgggcccctgctgcgcctagtggcagaggtggaggagagcccggagacagccgcccaccccgggcctatcgtagtccactgcagtgcagggattggccggacgggctgcttcatcgccacgcgaattggctgtcaacagctgaaagcccgaggagaagtggacattctgggtattgtgtgccaactgcggctagacagaggggggatgatccagacggcagagcagtaccagttcctgcaccacactttggccctgtatgcaggccagctgcctgaggaacccagcccctgacccctgccaccctccggtggcccaggtgcctacctccctcaagcctgggaaggtgggtctggggaaagtgggccgagtgatctgggggtacccttgggttggtgtggggaaggagtgcctccttagtggtgcttgacagtcacaggaagcagcagcagtaaggacaaggggccggattcaggtcttcaaccactggccactcctcttgccttcctctgttggccccagatggacagtaaggggaacctccaatgtctctctgaacttaaagacaggagctggcatttatgacagacaaagaaagaagcccaggtgtcctggtgttctctgagacactctttgtgatcttcagtttcctgttctataacatgaacataagtgcttagctgccatgagggaaaagtaatgagagaagttctagaagccactccagccactccttcctggggctgacaaaagggtgattccaagatcatccttcacccgaggtcctgcccaagcacaggccagatgcaagaatggggaaaagtctggtcctgatctccaagtctcaacatcctatcagtgactctgcctccctgaccacacatcggaagggcctggatgacccaatcaaaagaaagaacaaggactctggttacccttgcctccacccatgtgtcataagagtaggctacagaggtgaccaggcctggcagttgaaatctctggaagagggaacatgtggggactactcagaggcaaagaggagctgctcctgcctccatggttgctggccactcccaccaactactcttagggaggctaagcagtctctgttttgaccttccatggctcaataatacctggatgcaggaccactataccttgcatttgctgagtacacctagagagcttggctgtttccaaaaacaatcagggtcataaccatccatgcagacatggaggctcggctgaaccaggactcctcactgtctacctgagagaatgagcacccctcatccatctcagcatcaacacaatttccaggggacctcaggtctacctcaggactgaaccgccacacctcaggattcctcctccttgaatctgagactggctgcccattctgagatggggatgaaggtaagatgccgcatcaccagcacgccgcccctgacagctgccttgataccagctctctgtggaaacccccgaggagttggatctggagaacagctgggcctcctcactcaggacttctctcctgaagaacacgcagtgctaaaactgaggatgatttccctaatgcttctgcttggagtctcttatggaggagctgctccttccttacagcttggggatggacttcccacacctccacctcccctgagccctgagccctgtgagaggacgactgtctatgcaatgaggctcggtggggggctctcaagtgcctgatcctgcctggctcagaggcagccagagggaagcaactgacagccccacaggccctccctggcactgtccccatctcagagctcaggagggtacaagctccagaacagtaaccaagtgggaaaataaagacttcttggatgactg

SEQ ID NO: 6 - protein, 339 aa, Homo sapiens
  UNSURE (0)...(0): PTPN7 polypeptide sequence
MTQPPPEKTPAKKHVRLQERRGSNVALMLDVRSLGAVEPICSVNTPREVTLHFLRTAGHPLTRWALQRQPPSPKQLEEEFLKIPSNFVSPEDLDIPGHASKDRYKTILPNPQSRVCLGRAQSQEDGDYINANYIRGYDGKEKVYIATQGPMPNTVSDFWEMVWQEEVSLIVMLTQLREGKEKCVHYWPTEEETYGPFQIRIQDMKECPEYTVRHVTIQYQEERRSVKHILFSAWPDHQTPESAGPLLRLVAEVEESPETAAHPGPIVVHCSAGIGRTGCFIATRIGCQQLKARGEVDILGIVCQLRLDRGGMIQTAEQYQFLHHTLALYAGQLPEEPSP

SEQ ID NO: 7 - DNA, 3960 bp, Homo sapiens
  misc_feature (0)...(0): FEM-2 polynucleotide
ggacacggagccgcgaggagacagctgaggcccgcggagaccagggggtgaagcctggagaccctcttgccctggcctagctgcaggcccccgggatgctttgggcatgtcctctggagccccacagaagagcagcccaatggccagtggagctgaggagaccccaggcttcctggacacgctcctgcaagacttcccagccctgctgaacccagaggaccctctgccatggaaggccccagggacggtgctcagccaggaggaggtggagggcgagctggctgagctggccatgggctttctgggcagcaggaaggccccgccaccacttgctgctgctctggcccacgaagcagtttcacagctgctacagacagacctttccgaattcaggaagttgcccagggaggaagaagaagaggaggaggacgatgacgaggaggaaaaggcccctgtgaccttgctggatgcccaaagcctggcacagagtttctttaaccgcctttgggaagtcgccggccagtggcagaagcaggtgccattggctgcccgggcctcacagcggcagtggctggtctccatccacgccatccggaacactcgccgcaagatggaggaccggcacgtgtccctcccttccttcaaccagctcttcggcttgtctgaccctgtgaaccgcgcctactttgctgtgtttgatggtcacggaggcgtggatgctgcgaggtacgccgctgtccacgtgcacaccaacgctgcccgccagccagagctgcccacagaccctgagggagccctcagagaagccttccggcgcaccgaccagatgtttctcaggaaagccaagcgagagcggctgcagagcggcaccacaggtgtgtgtgcgctcattgcaggagcgaccctgcacgtcgcctggctcggggattcccaggtcattttggtacagcagggacaggtggtgaagctgatggagccacacagaccagaacggcaggatgagaaggcgcgcattgaagcattgggtggctttgtgtctcacatggactgctggagagtcaacgggaccctggccgtctccagagccatcggggatgtcttccagaagccctacgtgtctggggaggccgatgcagcttcccgggcgctgacgggctccgaggactacctgctgcttgcctgtgatggcttctttgacgtcgtaccccaccaggaagttgttggcctggtccagagccacctgaccaggcagcagggcagcgggctccgtgtcgccgaggagctggtggctgcggcccgggagcggggctcccacgacaacatcacggtcatggtggtcttcctcagggacccccaagagctgctggagggcgggaaccagggagaaggggacccccaggcagaagggaggaggcaggacttgccctccagccttccagaacctgagacccaggctccaccaagaagctaggtggtttccaggcccctgccctccccttcctcccatccttgtccttctctccctcagaagcctcaggacccaacaggtggcaggcagtggacagggtgcccgccccacagtgctttccccagcaccccagagccagtcgggacaccccccgcagcccgtcctggtggctgtggaactgcactgggtggcgggcagatggtggaaggcagcttaggagacctcaccaaagagaagatggaccggctcttgctcccagctcctattaggcccggggtgggaccagaggtcataggtgcccaacggcagccaaaccaaagacactggtgtgcatggggcagcatggttgtgcacgtgggaccctggggcggacccaggagccaaactcttgaagcaccccctgggtcaggcccagcagcggagtggccagccccagtttcccattgctcctctctgcggccagggccaggtgggttcatatttacagatatgcccagccagtcctggtcggccacaccagtgtcccaaagaggagagcgcagcagagccaggggtctgttctgtagcagccacccccctgcccccactccagggcagccatgatgtgcttggcccaccagggccttccgggctgctctcttccctgagcccggaaccggcgacgcacatgtgtcttttgttggtgtgtttgtttttttccagggaggtctaattccgaagcagtattccaggttttctctttgttttatcagtgccaagatgacctgttgtgtcatataatttaagcagagcttagcatttattttattctttagaaaacttaagtatttacttttttaaagctatttttcaaggaacctttttttgcagtattattgaatttattttctaaatcaggattgaaacaggaacttttccaggtggtgttaataagccattcaagtgccttacacagctttgaagaaactaggactgcagtgggctcggataggcccattgaggtttttagaaaagcaggatttgttttgttagggaggcatgattttggtgagatctttctggaagagttttccgcctctttgtgatgctgaacacccccaaggttctcccctccccccgctgcccaggtgactggcaggagctgcgactgccacgtagtgttgcctgggcccgacagcggggctctgggcatcccgggtgaccttggcccatctgcctgcattcccacccccttgggcctggctggatcccaggcagagggaccttgctgctgtgtgattggaacattcccaaatatcttgtgaatttgtaatcaaattggtctcattgggaaagactcttaattaagaggctcaggcaagcacagaggcagcccgtgggtctctgtctcagtctggaggcagcagggatgctgctgggagtccatggcacaggccacagcccctcaccttgccgcggtggctggcagcacgcctgccttgctctgccccatgccctgaacaggcatgagagctccacgtcccctagtgcaccctgagagggggctcacaagtgaccgatcctgggtgcctcagggagctcactgagggcgtgcaaagttgaaagtggcaaggctgggggagggtgtcgggtagagggaagagggcagggggctaggggaggactcagaggccatctgcagggccaagccacaggaagggctgagctggaggtgggcagggctgctccaggcaggtcagagcagtgcagggggaggagaggagaaagggaggaagctgggctgtgtggtccccatgaaggcattcagagtccacctgcagacagcgagagccccaggaaggtttgcacagctgtgccccaagcaccttggcctcctctcagctcgccgaggaggcacgctagagccgccttcccggtgggagccctctgtcccacagggagcggggagccagctttgctggggccctacctgcatgcccagccttacccctcattctcacagcacagatgaggttgagaccatgcagtcaatgcattgcttaaggtctcttatttacaaaaaaaaaccttaaacatagtcgctgtcattcagacattcagagaatggttggccacaaacaatgaccaagtattgcttggcttaacttgaaggcctgctgtctccttctgggggtcagggacgcagctccaccctcaccactagcccaccctgcccgtgggcataaccttgacgaagagagagaatgattggcatctgcttttctcttttctttgctaataattctgttcctggctgccgagagtgaagtttcaccatgtggaggtttggctcctatcacctggtggtctgattcataccctagcctgaggctccactggaagatctcgcagcctcagtgtatgggaaaccctttccccaggcttgtcccagcactgccgctccccacccctgagccaggaccccagaggatggccatgccccgtgcctggca

SEQ ID NO: 8 - protein, 454 aa, Homo sapiens
  UNSURE (0)...(0): FEM-2 polypeptide sequence
MSSGAPQKSSPMASGAEETPGFLDTLLQDFPALLNPEDPLPWKAPGTVLSQEEVEGELAELAMGFLGSRKAPPPLAAALAHEAVSQLLQTDLSEFRKLPREEEEEEEDDDEEEKAPVTLLDAQSLAQSFFNRLWEVAGQWQKQVPLAARASQRQWLVSIHAIRNTRRKMEDRHVSLPSFNQLFGLSDPVNRAYFAVFDGHGGVDAARYAAVHVHTNAARQPELPTDPEGALREAFRRTDQMFLRKAKRERLQSGTTGVCALIAGATLHVAWLGDSQVILVQQGQVVKLMEPHRPERQDEKARIEALGGFVSHMDCWRVNGTLAVSRAIGDVFQKPYVSGEADAASRALTGSEDYLLLACDGFFDVVPHQEVVGLVQSHLTRQQGSGLRVAEELVAAARERGSHDNITVMVVFLRDPQELLEGGNQGEGDPQAEGRRQDLPSSLPEPETQAPPRS

SEQ ID NO: 9 - DNA, 2786 bp, Homo sapiens
  misc_feature (0)...(0): DKFZP566K0524 polynucleotide
tggacccaactggcgaggctgctggggttgcagcgggacagttggggcggccccgcaggcccaggtttttgaaaataaagttaattcagagaaggtaaaactttctcttcggaatttcccacataatgattatgaggatgtttttgaagagccttcagaaagtggcagtgatcccagcatgtggacagccagaggccccttcagaagagacaggtggagcagtgaggatgaggaggctgcagggccatcacaggctctctcccctctactttctgatacgcgcaaaattgtttctgaaggagaactagatcagttggctcagattcggccattaatattcaattttcatgagcagacagccatcaaggattgtttgaaaatccttgaggaaaaaacagcagcgtatgatatcatgcaggaatttatggctttagaacttaagaatctgcctggtgagttctactctgggaatcaaccaagcaacagagaaaaaaacagataccgagatattcttccatatgattcaacacgcgttcctcttggaaaaagcaaggactacatcaatgctagttatattagaatagtcaattgtggagaagagtatttttatatcgctactcaaggaccactgctgagcaccatagatgacttttggcaaatggtgttggaaaataattcaaatgttattgccatgataaccagagagatggaaggtggaattatcaaatgctaccattactggcccatttctctgaagaagccattggaattgaaacacttccgtgtattcctggagaactaccagatacttcaatatttcatcattcgaatgtttcaagttgtggagaagtccacgggaactagtcactctgtaaaacagttgcagttcaccaagtggccagaccatggcactcctgcctcagcagatagcttcataaaatatattcgttatgcaaggaagagccaccttacaggacccatggttgttcactgcagtgccggcataggccggacaggggtgttcctatgtgtggatgtcgtgttctgtgccatcgtaaaggactgttcattcaacatcatggatatagtggcccaaatgagagaacaacgttctggcatggttcaaacgaaggagcagtatcacttttgttacgatattgtgcttgaagttcttcggaaacttctgactttggattaagaaagacttctgttgcctctcacttgaaattaccaagtgggtttgcacctcctcataaagaacatgtttgcactgtgctgaagggctttgctatgcatacaatctgctttcttggtttatcagtttattttctttctaaaagctccctgaagggcaatatcatttggcttggggtgatcagtgtttacttattgatcttgctagacaatatcaaaataacttcccacattttccagtgaaacagatgttacataaaacgattgcagcttggctatttggttgaagggattacagagcccaataaaggatttaaaatatattcattaagattttatttggaaaggtggctggagagagctgaggatttccaggactttgtaagttcttattctgggagaacataaggccaataatcatgacctcttccaggcatttttaagacagatgtctattcatgttctttagctagagcctgtactttttgctggcatttgaataacccagtttaaaaagagtccagttagggtggactaactttggacacaaattggcttccatttcctacattttcatactgctgccttcctacagctgctagaccaagacctgttggtctgggaagcatttcatggatagggagagctcctctcggtgaacagtccaaaactaaaatagatgtttatatagaaagcccaagaggagatttttgccatgcctgagttctttcctatcccaccctaacacttaacatattacttagtctgctttgttaaaagcaagtattacctttaacttgcctcttactctttgccctttagctaactaataaagtttgatatgggcattattatataattctgagtcattcatggtatctctcatgtttgatgtatttttcaaactaagatctatgatagtttttttttccagagttccatcaaatcatttatttcctttactttctcacctctgttgaaacatttagaaactggatttgggaacccaattttggaaaaccagattcatagtcatgaaaatggaaacttccatattctgtttttgaaaagatgtggccattattacagtaattttattataggactttgcctcgtacaattaatagtgatattttggacaaggagttctggtgacaagctatacctaattataagctataaaacaatagatatgagtgtttgtacagtttaactcaatggagatcagaatattctatgtattgagaaaatgtttaatatcaatctataaatcttgaatttctaagaggcttattttgttcttttggctgaatgagtatatttgaattggttgaataattaataattctcattgtaaaaataattatatgccaaaaatatatttgatgttaaatcaaatagatgattctgtttacattgttcatatgaataataatctgtgttaatttcattttgataattggcctttaatatttgtatctctaattttattttctctctgttactgtaaaataatagctataatgtataacaattttcttcagaagaattctatgctattattaaaataaaatatttactgaaaaaaaaaa

SEQ ID NO: 10 - protein, 398 aa, Homo sapiens
  UNSURE (0)...(0): DKFZP566K0524 polypeptide
GPNWRGCWGCSGTVGAAPQAQVFENKVNSEKVKLSLRNFPHNDYEDVFEEPSESGSDPSMWTARGPFRRDRWSSEDEEAAGPSQALSPLLSDTRKIVSEGELDQLAQIRPLIFNFHEQTAIKDCLKILEEKTAAYDIMQEFMALELKNLPGEFYSGNQPSNREKNRYRDILPYDSTRVPLGKSKDYINASYIRIVNCGEEYFYIATQGPLLSTIDDFWQMVLENNSNVIAMITREMEGGIIKCYHYWPISLKKPLELKHFRVFLENYQILQYFIIRMFQVVEKSTGTSHSVKQLQFTKWPDHGTPASADSFIKYIRYARKSHLTGPMVVHCSAGIGRTGVFLCVDVVFCAIVKDCSFNIMDIVAQMREQRSGMVQTKEQYHFCYDIVLEVLRKLLTLD

SEQ ID NO: 11 - DNA, 2226 bp, Homo sapiens
  misc_feature (0)...(0): FLJ20313 nucleotide sequence
ctcctctgcgcttccgtggagcctccaggccgacccccgggaactggaggaccccaggaggctgcgcgcgtctccctgcccacagcagcgcggctgcctgattcccggcgccgcgaaatgcgccttctcgggagcccccactggctcggcgaaaacttactgacgataagatcaattcggaaccgaagattaaaaaactggagccagtccttttgccaggagaaattgtcgtaaatgaagtcaattttgtgagaaaatgcattgcaacagacacaagccagtacgatttgtggggaaagctgatatgcagtaacttcaaaatctcctttattacagatgacccaatgccattacagaaattccattacagaaaccttcttcttggtgaacacgatgtccctttaacatgtattgagcaaattgtcacagtaaacgaccacaagaggaagcagaaagtcctaggccccaaccagaaactgaaatttaatccaacagagttaattatttattgtaaagatttcagaattgtcagatttcgctttgatgaatcaggtcccgaaagtgctaaaaaggtatgccttgcaatagctcattattcccagccaacagacctccagctactctttgcatttgaatatgttgggaaaaaataccacaattcagcaaacaaaattaatggaattccctcaggagatggaggaggaggaggaggaggaggtaatggagctggtggtggcagcagccagaaaactccactctttgaaacttactcggattgggacagagaaatcaagaggacaggtgcttccgggtggagagtttgttctattaacgagggttacatgatatccacttgccttccagaatacattgtagtgccaagttctttagcagaccaagatctaaagatcttttcccattcttttgttgggagaaggatgccactctggtgctggagccactctaacggcagtgctcttgtgcgaatggccctcatcaaagacgtgctgcagcagaggaagattgaccagaggatttgtaatgcaataactaaaagtcacccacagagaagtgatgtttacaaatcagatttggataagaccttgcctaatattcaagaagtacaagcagcatttgtaaaactgaagcagctatgcgttaatgagccttttgaagaaactgaagagaaatggttatcttcactggaaaatactcgatggttagaatatgtaagggcattccttaagcattcagcagaacttgtatacatgctagaaagcaaacatctctctgtagtcctacaagaggaggaaggaagagacttgagctgttgtgtagcttctcttgttcaagtgatgctggatccctattttaggacaattactggatttcagagtctgatacagaaggagtgggtcatggcaggatatcagtttctagacagatgcaaccatctaaagagatcagagaaagagtctcctttatttttgctattcttggatgccacctggcagctgttagaacaatatcctgcagcttttgagttctccgaaacctacctggcagtgttgtatgacagcacccggatctcactgtttggcaccttcctgttcaactcccctcaccagcgagtgaagcaaagcacggtcagtaggataaaaagttgtacaaaacaagattattttccttcacgagtttgaagtttctggtcacaattcattgatgtagaggatttatgactaagcagggtctcaagccaaacttgaaaccattctgaaccaaagtgccatttcacccacctcgaaccaacaacagaagctgacaaatgccgtggagaccattgagggaaacagaaaggggcagctcttgtggaccttcaggaagcctttctaggaagaggattgccctcatagtgagctccggggtcttcagcctcagccgtaaggccctgggctaggcagtgtgacctagggagcgggaaacctgagttctggccctggtctgggaaaagtgctaggcccatgttccactcaggcttcagcctgagagtccaggttgctaacctgtaaaatggatctgtcaaactaacacttatgcctttagtctcattgtatgaggtgtaacattttgtaaactgtgaatcattatgcaaattttcctaaagacatatgaattattctggatttgttggtataaaagacaaaatacactggtcaaaaaaaaagt

SEQ ID NO: 12 - protein, 451 aa, Homo sapiens
  UNSURE (0)...(0): FLJ20313 polypeptide sequence
MPLQKFHYRNLLLGEHDVPLTCIEQIVTVNDHKRKQKVLGPNQKLKFNPTELIIYCKDFRIVRFRFDESGPESAKKVCLAIAHYSQPTDLQLLFAFEYVGKKYHNSANKINGIPSGDGGGGGGGGNGAGGGSSQKTPLFETYSDWDREIKRTGASGWRVCSINEGYMISTCLPEYIVVPSSLADQDLKIFSHSFVGRRMPLWCWSHSNGSALVRMALIKDVLQQRKIDQRICNAITKSHPQRSDVYKSDLDKTLPNIQEVQAAFVKLKQLCVNEPFEETEEKWLSSLENTRWLEYVRAFLKHSAELVYMLESKHLSVVLQEEEGRDLSCCVASLVQVMLDPYFRTITGFQSLIQKEWVMAGYQFLDRCNHLKRSEKESPLFLLFLDATWQLLEQYPAAFEFSETYLAVLYDSTRISLFGTFLFNSPHQRVKQSTVSRIKSCTKQDYFPSRV